(19) United States
(12) Patent Application Publication
(10) Pub. No.: US 2026/0286381 A1
(43) Pub. Date: Sep. 24, 2026

(54) BACTERIAL GLUTAMINE SYNTHETASE AS SELECTION MARKER IN MAMMALIAN CELLS

(71) Applicant: Boehringer Ingelheim International GmbH, Ingelheim am Rhein (DE)

(72) Inventors: Moritz SCHMIDT, Erbach (DE); Daniel HEINZELMANN, Ulm (DE); Franziska REUSS, Ringschnait (DE); Simon FISCHER, Staig (DE); Patrick SCHULZ, Warthausen (DE)

(21) Appl. No.: 18/848,530

(22) PCT Filed: Mar. 22, 2023
(86) PCT No.: PCT/EP2023/057369

§ 371 Date: Sep. 19, 2024

(30) Foreign Application Priority Data
Mar. 23, 2022 (EP) 22163849.7

(51) Int. Cl. C12N 15/85; C12N 5/071; C12N 9/00; C12P 21/00

(52) U.S. Cl. CPC C12N 15/85; C12N 5/0602; C12N 9/93; C12P 21/00; C12Y 603/01002; C12N 2510/02; C12N 2830/34

(57) ABSTRACT

The invention relates to a mammalian expression vector comprising a polynucleotide encoding a bacterial glutamine synthetase as a selection marker and to a nucleic acid and a mammalian host cell encoding said bacterial glutamine synthetase. Particularly, the bacterial glutamine synthetase is derived from bacteria of the order Enterobacterales and the family of Morganellaceae, preferably the bacterial glutamine synthetase is a Providencia species glutamine synthetase, optionally further comprises a mutation in position E130 and/or F226 and/or R345A. The invention further relates to methods for preparing a mammalian cell stably expressing a protein of interest and/or a non-coding RNA and to in a method for producing a protein in a mammalian cell using said bacterial glutamine synthetase.

### RELATED APPLICATION DISCLOSURE

This Application is a National Stage Application of International Application No. PCT/EP2023/057369, filed Mar. 22, 2023, which is hereby incorporated by reference herein in its entirety and which claims the foreign priority benefit of European Patent Application No. 22163849.7, filed Mar. 23, 2022.

### SEQUENCE DISCLOSURE

This application includes, as part of its disclosure, a “Sequence Listing XML” pursuant to 37 C.F.R. § 1.83l(a) which is submitted in XML file format via the USPTO patent electronic filing system in a file named 01-3500-US-1-2026-01-30.xml, created on Jan. 30, 2026, and having a size of 53,643 bytes, which is hereby incorporated by reference herein in its entirety.

### FIELD OF THE INVENTION

The invention relates to a mammalian expression vector comprising a polynucleotide encoding a bacterial glutamine synthetase as a selection marker with a sequence identity of at least 85% with the amino acid sequence of SEQ ID NO: 1 and to a nucleic acid and a mammalian host cell encoding said bacterial glutamine synthetase. Particularly, the bacterial glutamine synthetase is derived from bacteria of the order Enterobacterales and of the family of Morganellaceae, preferably the bacterial glutamine synthetase is a Providencia species glutamine synthetase, optionally further comprises a mutation in position E130 and/or F226 and/or R345. The invention further relates to methods for preparing a mammalian cell stably expressing a protein of interest and/or a non-coding RNA and to a method for producing a protein in a mammalian cell using said bacterial glutamine synthetase.

### BACKGROUND

Chinese hamster ovary (CHO) cells are one of the most commonly employed mammalian cell lines used for the production of therapeutic proteins, such as antibodies. An important aspect is to generate productive and stable cell lines expressing the protein of interest in a short period of time and at high product titers with suitable product quality. To produce a stable and high-producing cell line a selection phase is required to generate stable heterogenous cell pools consisting of different clones which need to be isolated and screened, before the final production cell line can be preserved in a cell bank. Metabolic selection systems such as the dihydrofolate reductase (DHFR) and the glutamine synthetase (GS) selection system are commonly used to improve this process and to generate stable cell lines more efficiently.

In cell lines deficient of the DHFR gene, such as CHO-DG44 cells, selection is performed in the absence of hypoxanthine and thymidine in the medium. Amplification steps by adding increasing concentration of methotrexate (MTX) may be used in addition. The GS selection system is advantageous as it requires fewer gene copies for the survival and hence selection is faster for high producing cell pools.

Glutamine synthetase (EC 6.3.1.2, also known as γ-glutamyl: ammonia ligase) catalyzes ATP-dependent condensation of ammonia and glutamate to form glutamine. Glutamine synthetases are classified in three subgroups: GSI, GSII and GSIII. The CHO GS is a class II enzyme, the subclass predominantly expressed by eukaryotes, whereas bacterial GS proteins are typically members of the GSI class. While GSI and GSII catalyze the same reaction, they show no or very little sequence similarity except for the residues forming part of the active site and are overall rather different. For example, bacterial type I GS is a 12-subunit complex (Eisenberg et al. (2000), Biochim. Biophys. Acta 1477, 122-145), and GSII has been reported to form two or three pentameric ring stackings (Krajewski et al. (2008), J. Mol. Biol. 375, 217-228). Also, bacterial GS may have very little sequence similarity with each other, as low as about 50% amino acid identity or less of e.g., Bacillus coagulans (CN 12625930 A) or Mycobacterium tuberculosis (WO 2006/000045) or Cornybacterium glutamicum (CN 1884501 A) with Providencia vermicola GS (Zuo et al., Scientific Reports, 2018, 8 (1): 1-8 and supplementary material).

GS is an ubiquitous enzyme essential for nitrogen metabolism. Thus, GS has been used as selection marker that is introduced via a mammalian expression construct. In cell lines that do not express sufficient levels of endogenous GS, removal of glutamine supplementation from the cell culture media increases the selection pressure on cells. In cell lines having insufficient endogenous GS levels, such as mouse myeloma cell lines, culturing in the absence of glutamine or lack of glutamine supplementation provides sufficient selection pressure to isolate stable recombinant cell lines. In cell lines having sufficient endogenous GS, such as CHO cells, addition of the GS inhibitor methionine sulfoximine (MSX) or generation of GS knockout cells (GS−/− or GS−/+) are required to allow sufficient selection pressure in the absence of glutamine to isolate productive cell lines. Selection stringency in CHO GS knockout cells is strongly improved and expression of the transfected GS gene under the control of a weak promoter has further been reported to improve selection stringency with or without the use of MSX. In addition to selection stringency and productivity, the stability of protein production of high producing clones has been shown to be a critical attribute.

Also, the transfected glutamine synthetase selection marker has substantial influence on the selection process and the phenotypic stability as well as productivity of CHO-based cell lines. For example, the use of attenuated variants of the CHO glutamine synthetase was further shown to improve stability, selection behavior and productivity (Lin et al. (2019), mAbs, 11:5, 965-976; WO 2018/093331, US20190352631, WO 2017/197098). Most of the described attenuated variants had mutations in the conserved substrate-binding residues. For instance, two attenuated GS mutants containing R324C and R341C mutations were first identified in two unrelated infants with congenital GS deficiency (Lin et al. (2019), mAbs, 11:5, 965-976). The residues reported to be involved in binding of glutamate are E134, E136, E196, E203, N248, G249, H253, R299, R319, E338 and R340; the residues reported to be involved in binding of ATP are W130, A191, G192, P208, N255, S257, R262, R324 and Y336; and the residues to be involved in binding of ammonia are D63, S66, D162 and E305 (amino acid numbering refers to human GS) (Krajewski et al. (2008), J. Mol. Biol. 375, 217-228 and WO 2018/093331).

Although attenuated GS variants harboring mutations in the active site show an increased selection stringency, the duration of the selection process and the overall cell growth characteristics can be impaired, negatively affecting bioprocess performance. Attenuation of the selection marker is a fine balance between selection stringency, integrated copy numbers and suitable cell culture performance (e.g. growth behavior) to support the manufacturing of recombinant proteins. Thus, there is a need to discover novel attenuated GS variants to improve mammalian expression vectors in cell line development.

Although bacterial GS or peptides thereof from various species have been expressed in eukaryotic cells such as yeast, for purification, as immunogen or other reasons (see e.g., WO 2018/144807, WO 2006/000045 A1) and yeast or plant GS was shown to be active in E. coli but not vice versa (WO 2004/003175 A2 and EP 0 240 792 A1), bacterial GS (GSI) were assumed not to be active in mammalian cells. More importantly, to the best of our knowledge bacterial GS have not been tested or used in mammalian cells as selection marker before.

### SUMMARY OF THE INVENTION

The present invention demonstrates that certain bacterial glutamine synthetase genes, unrelated to mammalian glutamine synthetase genes, can be used to generate highly productive and stable mammalian host cell pools expressing a therapeutic protein, particularly a therapeutic antibody, showing a more stringent selection behavior due to the attenuated activity compared to wildtype glutamine synthetase and beneficial cell culture performance in mammalian cells such as CHO cells. This is the first time that prokaryotic glutamine synthetase variants were used to generate stable and productive mammalian cell pools for cell line development. Further, point mutations of Providencia vermicola glutamine synthetase were identified that exhibit extended selection stringency and improved antibody titers compared to wildtype Providencia vermicola glutamine synthetase. Another advantage of a bacterial GS selection marker is that due to the low sequence similarity to mammalian (e.g., CHO) GS the probability of a recombination event that restores the endogenous GS gene and hence enables survival of cells not producing the gene of interest is dramatically reduced.

In a first aspect the invention relates to a mammalian expression vector comprising a polynucleotide encoding a bacterial glutamine synthetase as a selection marker, wherein the bacterial glutamine synthetase has at least 85% sequence identity with the amino acid sequence of SEQ ID NO: 1. In certain embodiments the bacterial glutamine synthetase is derived from bacteria of the order Enterobacterales and of the family of Morganellaceae. In certain embodiments, the bacterial glutamine synthetase is derived from a Providencia species and/or comprises an amino acid sequence having at least 95% sequence identity with amino acid sequence of SEQ ID NO: 1. In certain embodiments the bacterial glutamine synthetase is derived from a Photorhabdus species and/or comprises an amino acid sequence having at least 95% sequence identity with amino acid sequence of SEQ ID NO: 14. In a preferred embodiment the bacterial glutamine synthetase is a Providencia vermicola glutamine synthetase or a Photorhabdus luminescens glutamine synthetase.

The mammalian expression vector may further encode the bacterial glutamine synthetase comprising an amino acid sequence having at least 85% sequence identity with the amino acid sequence of SEQ ID NO: 1, and a mutation selected from the group consisting of E130X, F226Y, R345A and combinations thereof, wherein X is any amino acid, preferably wherein the mutation in amino acid position E130 is a substitution with an aromatic or hydrophobic amino acid, more preferably selected from the group consisting of Y, W, F, A, G, V, L, M and I. In certain embodiments the amino acid sequence has at least 95% sequence identity with the amino acid sequence of SEQ ID NO: 1 or SEQ ID NO: 14, and a mutation selected from the group consisting of E130X, F226Y, R345A and combinations thereof, wherein X is any amino acid, preferably wherein the mutation in amino acid position E130 is a substitution with an aromatic or hydrophobic amino acid, more preferably selected from the group consisting of Y, W, F, A, G, V, L, M and I.

In certain embodiments, the mammalian expression vector further comprises at least one polynucleotide encoding a protein of interest and/or a non-coding RNA, preferably an expression cassette comprising at least one polynucleotide encoding a protein of interest and/or a non-coding RNA. The protein of interest is preferably a therapeutic protein, more preferably selected from the group consisting of a cytokine, a hormone, a fusion protein, an antibody, an antibody-derived molecule and an antibody mimetic.

The invention further provides a nucleic acid sequence comprising a polynucleotide encoding a bacterial glutamine synthetase operably linked to a mammalian promoter, optionally further comprising at least one polynucleotide encoding a protein of interest and/or a non-coding RNA. In certain embodiments the bacterial glutamine synthetase comprises an amino acid sequence having at least 85% sequence identity with the amino acid sequence of SEQ ID NO: 1, and a mutation selected from the group consisting of E130X, F226Y, R345A and combinations thereof, wherein X is any amino acid, preferably wherein the mutation in amino acid position E130 is a substitution with an aromatic or hydrophobic amino acid, more preferably selected from the group consisting of Y, W, F, A, G, V, L, M and I.

In yet another aspect, the invention provides a bacterial glutamine synthetase from Providencia vermicola comprising an amino acid sequence having at least 85% sequence identity with the amino acid sequence of SEQ ID NO: 1 and a mutation selected from the group consisting of E130X, F226Y, R345A and combinations thereof, wherein X is any amino acid, preferably wherein the mutation in amino acid position E130 is a substitution with an aromatic or hydrophobic amino acid, more preferably selected from the group consisting of Y, W, F, A, G, V, L, M and I.

In yet another aspect, the invention provides a mammalian host cell comprising the expression vector according to the invention, the nucleic acid sequence according to the invention or a nucleic acid sequence encoding the bacterial glutamine synthetase according to the invention, preferably wherein the mammalian host cell is (a) a rodent cell, more preferably a CHO cell and/or (b) a GS gene knockout cell. Preferably, the polynucleotide encoding the bacterial glutamine synthetase is co-integrated with at least one polynucleotide encoding a protein of interest and/or a non-coding RNA into the host cell genome.

In yet another aspect, the invention provides a method for preparing a cell stably expressing a protein of interest and/or a non-coding RNA, comprising (a) introducing the expression vector according to the invention or the nucleic acid according to the invention comprising a polynucleotide encoding a bacterial glutamine synthetase and at least one polynucleotide encoding a protein of interest and/or a non-coding RNA into a mammalian host cell, preferably into a CHO cell; and (b) culturing the mammalian host cell in a medium comprising no glutamine under conditions to select for the bacterial glutamine synthetase, wherein the at least one polynucleotide encoding a protein of interest and/or a non-coding RNA is co-integrated with the polynucleotide encoding the bacterial glutamine synthetase into the host cell genome. In certain embodiments the method further comprises (c) a step of isolating a single clone for clonal expansion to prepare a monoclonal cell line. In such case the method is a method for preparing a monoclonal cell line stably expressing a protein of interest and/or a non-coding RNA.

The invention further provides a method of producing a protein of interest, comprising (a) introducing the mammalian expression vector according to the invention or the nucleic acid according to the invention into a mammalian host cell, preferably into a CHO cell, wherein the expression vector comprises a polynucleotide encoding a bacterial glutamine synthetase operably linked to a mammalian promoter and at least one polynucleotide encoding a protein of interest; (b) culturing the mammalian host cell in a medium comprising no glutamine under conditions to select for the bacterial glutamine synthetase, wherein the at least one polynucleotide encoding a protein of interest is co-integrated with the polynucleotide encoding the bacterial glutamine synthetase into the host cell genome; (c) optionally isolating a single clone for clonal expansion to prepare a monoclonal cell line; (d) culturing the mammalian host cell under conditions to produce the protein of interest; and (e) harvesting and optionally purifying the protein of interest.

In yet another aspect, a method of producing a protein of interest is provided comprising (a) providing the mammalian host cell of the invention, comprising a polynucleotide encoding a bacterial glutamine synthetase operably linked to a mammalian promoter and at least one polynucleotide encoding a protein of interest; (b) culturing the mammalian host cell under conditions to produce the protein of interest; and (c) harvesting and optionally purifying the protein of interest. Preferably the mammalian host cell is a rodent cell, more preferably a CHO cell.

In yet another aspect, the invention relates to a kit comprising the expression vector according to the invention and a cell culture medium not comprising glutamine.

In yet another aspect, the invention provides a use of a bacterial glutamine synthetase as a selection marker in mammalian cells.

### DESCRIPTION OF THE FIGURES

FIG. 1: CHO-K1-GS cell pools expressing mAb1 and wildtype CHO GS as selection marker. CHO-K1-GS cell pools stably transfected with a mammalian expression vector to express mAb1 and CHO wildtype GS as a metabolic selection maker (n=5) were cultured in medium not containing L-glutamine. Titer [mg/L] of mAb1 was determined at the indicated days post transfection.

FIG. 2: Viability and viable cell density (VCD) of CHO-K1-GS expressing mAb1 and wildtype CHO GS or Providencia vermicola GS as selection marker. CHO-K1-GS cell pools stably transfected with a vector encoding the transcription cassette of mAb1 and glutamine synthetase selection marker from Cricetulus griseus or Providencia vermicola were cultured in medium not containing L-glutamine. A) Viability [%] and B) viable cell density during selection was determined at the indicated days post transfection.

FIG. 3: Productivity post selection and shake-flask production run of CHO-K1-GS expressing mAb1 and wildtype CHO GS or Providencia vermicola GS as selection marker. CHO-K1-GS cell pools stably transfected with a vector encoding the transcription cassette of mAb1 and glutamine synthetase selection marker from Cricetulus griseus (CHO WT GS) or Providencia vermicola were cultured in medium not containing L-glutamine. Productivity as titer [mg/L] was determined (A) post-selection at the indicated days post transfection using CHO WT GS or P. vermicola GS as selection marker and (B) in a shake-flask production run at the indicated days post transfection for P. vermicola GS only, because CHO WT GS cells lost their productivity after about 20 days.

FIG. 4: Viability and productivity post transfection of CHO-K1-GS expressing mAb1 and GS from Providencia vermicola, Photorhabdus luminescens and Budvicia aquatica as selection marker. CHO-K1-GS cell pools stably transfected with a vector encoding the transcription cassette of mAb1 and glutamine synthetase selection marker from Providencia vermicola (filled circle), Photorhabdus luminescens (filled square) and Budvicia aquatica (filled triangle) were cultured in medium not containing L-glutamine. A) Viability [%] during selection was determined at the indicated days post transfection. (B) Productivity as titer [mg/L] was determined post-selection at the indicated days post transfection.

FIG. 5: Shake-flask production run of CHO-K1-GS expressing mAb1 and wildtype Providencia vermicola GS or Providencia vermicola GS E130F/E130G mutants as selection marker. CHO-K1-GS cell pools stable transfected with a transcription cassette to express mAb1 and Providencia vermicola GS or Providencia vermicola GS harboring a E130F or E130G mutation as a selection marker were cultured in medium not containing L-glutamine. Titer [mg/L] of mAb1 was determined at the indicated days post transfection.

FIG. 6: Productivity and viable cell density (VCD) in shake-flask production run of CHO-K1-GS expressing mAb1 and Providencia vermicola wildtype GS, CHO wildtype GS or Providencia vermicola F226Y variant as selection marker. After stable pool generation and single cell deposition, selected high producing clones were subjected to shaking flask experiment (14 days production run) head-to-head with CHO WT. (A) Productivity as mAb1 titer [mg/L) using of Providencia vermicola wildtype GS, Providencia vermicola GS F226Y or CHO GS WT as selection marker and (B) viable cell density [1×10⁶ cells/ml] of Providencia vermicola wildtype GS and Providencia vermicola GS F226Y as selection marker were determined at the end of production run.

FIG. 7: Viability and productivity post transfection of CHO-K1-GS expressing mAb1 and Providencia vermicola wildtype GS or Providencia vermicola R345A or R360A variants as selection marker. CHO-K1-GS cell pools stably transfected with a vector encoding the transcription cassette of mAb1 and glutamine synthetase selection marker from Providencia vermicola WT GS (filled circle), and Providencia vermicola R345A (filled square) or R360A variants (filled triangle) were cultured in medium not containing L-glutamine. A) Viability [%] during selection was determined at the indicated days post transfection for Providencia vermicola wildtype GS and the two variants. (B) Productivity as titer [mg/L] was determined post-selection at the indicated days normalized to >70% viability for Providencia vermicola wildtype GS and Providencia vermicola R345A variant, because Providencia vermicola R360A variant did not recover during selection.

FIG. 8: Sequence of Providencia vermicola glutamine synthetase (SEQ ID NO: 1)

FIG. 9: Sequence of wild type Cricetulus griseus glutamine synthetase (SEQ ID NO: 2)

FIG. 10: Sequence of Photorhabdus luminescens glutamine synthetase (SEQ ID NO: 14)

### DETAILED DESCRIPTION

The term “comprises” or “comprising” means “including, but not limited to”. The term is intended to be open-ended, to specify the presence of any stated features, elements, integers, steps or components, but not to preclude the presence or addition of one or more other features, elements, integers, steps, components or groups thereof. The term “comprising” thus includes the more restrictive terms “consisting of” and “essentially consisting of”. With regard to sequences the terms “having an amino acid sequence of” and “comprising an amino acid of” are used interchangeably and include the embodiment “consisting of the amino acid sequence of”. Similarly the term “encoding” or “encodes” is intended to be open-ended and allows the presence or addition or one or more other features, elements or components. Furthermore, singular and plural forms are not used in a limiting way. As used herein, the singular forms “a”, “an” and “the” designate both the singular and the plural, unless expressly stated to designate the singular only.

The term “protein” is used interchangeably with “amino acid sequence” or “polypeptide” and refers to polymers of amino acids of any length. These terms also include proteins that are post-translationally modified through reactions that include, but are not limited to, glycosylation, acetylation, phosphorylation, glycation or protein processing. Modifications and changes, for example fusions to other proteins, amino acid sequence substitutions, deletions or insertions, can be made in the structure of a polypeptide while the molecule maintains its biological functional activity. For example, certain amino acid sequence substitutions can be made in a polypeptide or its underlying nucleic acid coding sequence and a protein can be obtained with the same properties.

The term “nucleic acid sequence” is used interchangeably with “polynucleotide” and refers to DNA or RNA of any length. In the context of an expression vector, particularly a plasmid, and integration into the host cell's genome the person skilled in the art would understand that it refers to a DNA sequence or molecule.

The term “eukaryotic cell” as used herein refers to cells that have a nucleus within a nuclear envelop and include animal cells, human cells, plant cells and yeast cells. Eukaryotic cell particularly encompasses mammalian cell, such as Chinese hamster ovary (CHO) cell or HEK293 cell derived cells. Mammalian cells as used herein refer to all cells or cell lines of mammalian origin, such as human or rodent cells. Cells as referred to herein are cells maintained in culture and do not relate to primary cells, but cell lines or cell line derived cells, i.e., to immortalized cells.

The term “about” as used herein refers to a variation of 10% of the value specified, for example, about 50% carries a variation from 45 to 55%.

The term “selection stringency” as used herein refers to the duration to reach more than 70% viability and a doubling time of 48 h or less of the cell culture following transfection. The longer the time period, the more stringent the selection behavior. Typically, an attenuated glutamine synthetase shows a more stringent selection behavior compared to CHO wildtype glutamine synthetase.

### Bacterial Glutamine Synthetase and Mammalian Expression Vector or Host Cells Encoding the Bacterial Glutamine Synthetase

The present invention demonstrates that certain bacterial glutamine synthetase genes, unrelated to mammalian glutamine synthetase genes, can be used as selection marker to generate highly productive and stable mammalian host cell pools or cell lines expressing a therapeutic protein, such as a therapeutic antibody, and/or a non-coding RNA, such as an siRNA or miRNA. This is the first time that prokaryotic glutamine synthetase variants have been successfully used as selection marker in mammalian cells and surprisingly the glutamine synthetase from Providencia vermicola as well as from Photorhabdus luminescens shows a more stringent selection behavior due to the attenuated activity compared to CHO wildtype glutamine synthetase in CHO cells and resulted in increased productivity compared to the use of CHO wildtype glutamine synthetase as selection marker in CHO cells. Having provided the proof-of-principle that certain bacterial glutamine synthetase genes can be used as selection markers in mammalian cells, further bacterial glutamine synthetase genes can be identified. Further, point mutations of Providencia vermicola glutamine synthetase in highly conserved amino acids were identified that exhibit extended selection stringency and improved antibody titers compared to wildtype Providencia vermicola glutamine synthetase. Due to the conservation of these amino acids in at least Photorhabdus luminescens glutamine synthetase, it is expected that these point mutations in Photorhabdus luminescens glutamine synthetase similarly exhibit extended selection stringency and improved antibody titers compare to wildtype Photorhabdus luminescens glutamine synthetase.

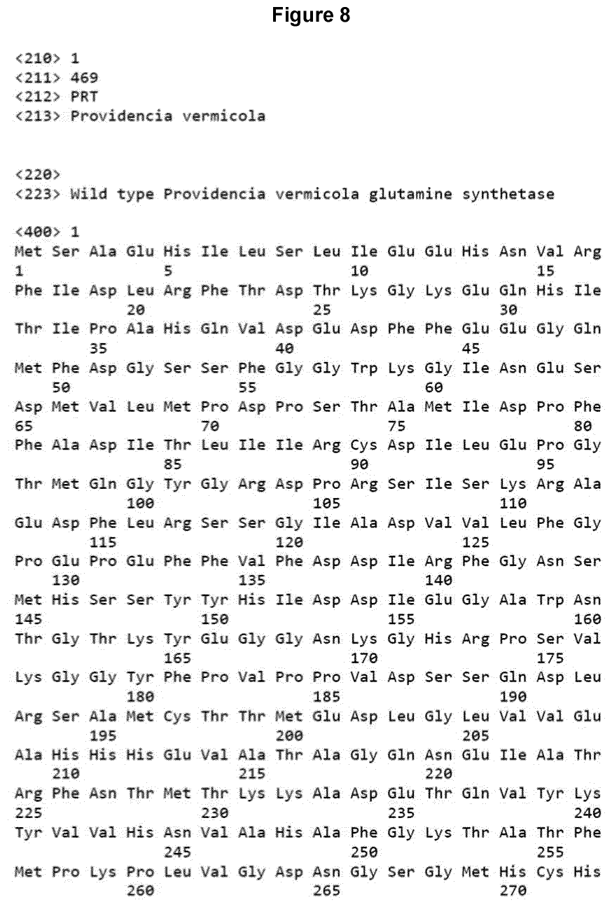

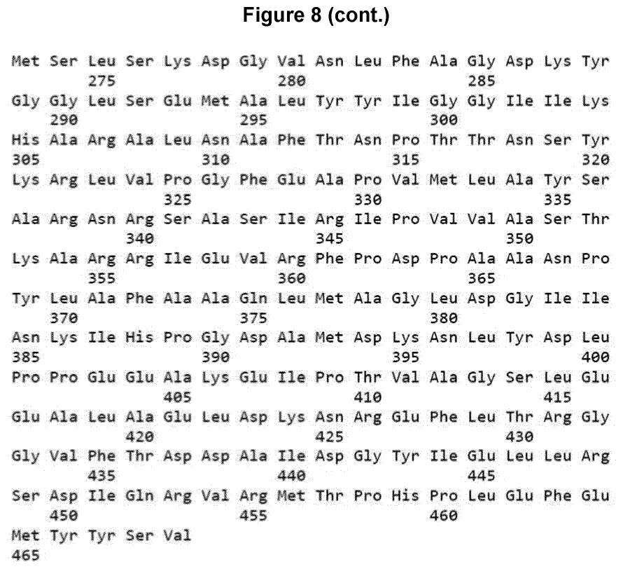

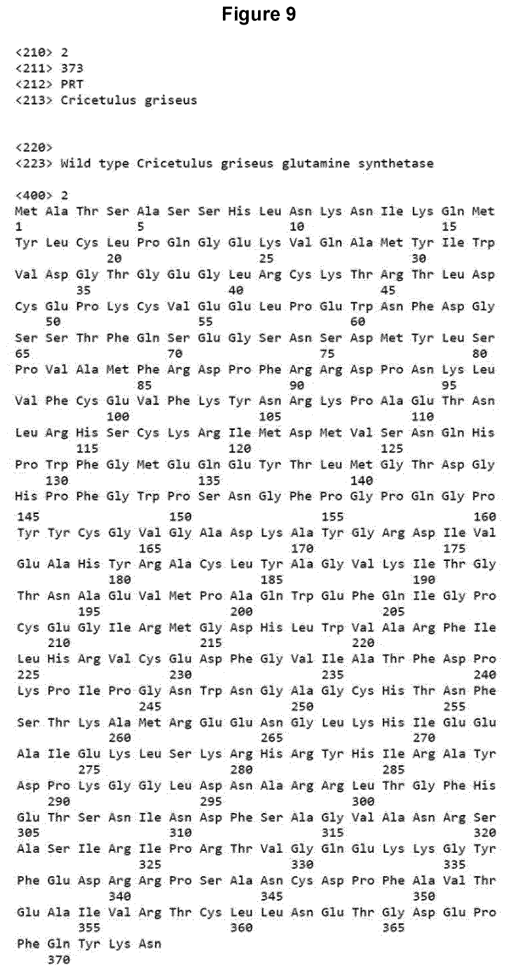

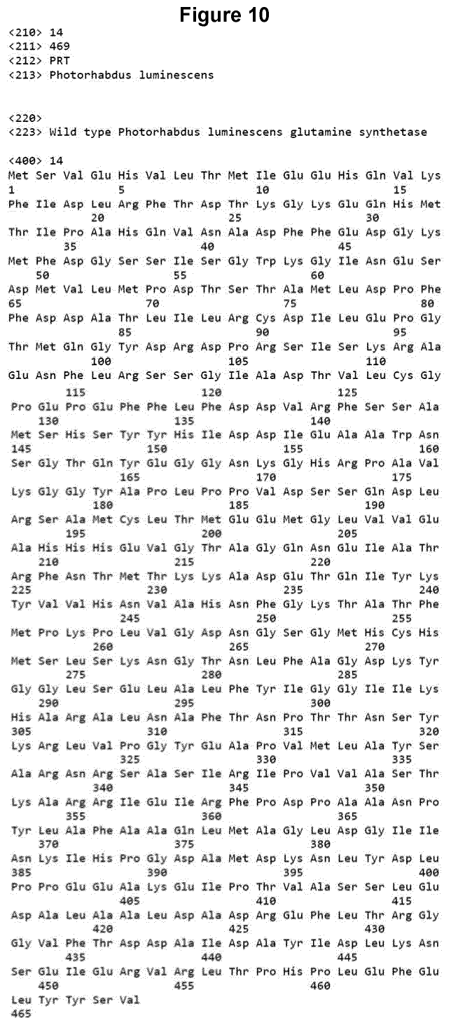

More specifically, in a first aspect the invention relates to a mammalian expression vector comprising a polynucleotide encoding a bacterial glutamine synthetase as a selection marker, wherein the bacterial glutamine synthetase comprises an amino acid sequence having at least 85% sequence identity with the amino acid sequence of SEQ ID NO: 1 (FIG. 8). In certain embodiments the bacterial glutamine synthetase is derived from bacteria of the order Enterobacterales and of the family of Morganellaceae. Upon transfection or transduction of the mammalian expression vector according to the invention, the bacterial glutamine synthetase mediates increased selection stringency and/or genetic stability of a stably co-integrated protein of interest and/or non-coding RNA in CHO cells compared to a CHO glutamine synthetase having the amino acid sequence of SEQ ID NO: 2 (FIG. 9). In certain embodiments, the bacterial glutamine synthetase is derived from a Providencia species and/or comprises an amino acid sequence having at least 95% sequence identity with amino acid sequence of SEQ ID NO: 1 (FIG. 8). In certain embodiments the bacterial glutamine synthetase is derived from a Photorhabdus species and/or comprises an amino acid sequence having at least 95% sequence identity with amino acid sequence of SEQ ID NO: 14 (FIG. 10). In a preferred embodiment the bacterial glutamine synthetase is a Providencia vermicola glutamine synthetase or a Photorhabdus luminescens glutamine synthetase. Preferably the glutamine synthetase is from a Providencia species, more preferably from Providencia vermicola. Thus, the glutamine synthetase may be a Providencia vermicola glutamine synthetase, particularly a bacterial glutamine synthetase comprising an amino acid sequence of SEQ ID NO: 1 or an amino acid sequence having at least 95% sequence identity with the amino acid sequence of SEQ ID NO: 1. In certain embodiments the bacterial glutamine synthetase comprises an amino acid sequence having at least 85%, at least 90% and preferably at least 95% sequence identity with SEQ ID NO: 1. In another embodiment, the bacterial glutamine synthetase comprises an amino acid sequence having at least 95%, at least 96%, at least 97%, at least 98%, at least 99% or 100% sequence identity with the amino acid sequence of SEQ ID NO: 1 or the bacterial glutamine synthetase comprises an amino acid sequence having at least 95%, at least 96%, at least 97%, at least 98%, at least 99% or 100% sequence identity with the amino acid sequence of SEQ ID NO: 14, preferably the bacterial glutamine synthetase comprises an amino acid sequence having at least 95%, at least 96%, at least 97%, at least 98%, at least 99% or 100% sequence identity with the amino acid sequence of SEQ ID NO: 1. Thus, the bacterial glutamine synthetase may have the amino acid sequence of SEQ ID NO: 1 or SEQ ID NO: 14.

The bacterial glutamine synthetase may further comprise point mutations that further attenuate the glutamine synthetase (e.g., E130X and/or R345A) or that increase catalytic activity (e.g., F226Y). Particularly, the bacterial glutamine synthetase may have a point mutation in a highly conserved residue corresponding to position E130 of Providencia vermicola glutamine synthetase having the amino acid sequence of SEQ ID NO: 1. The point mutation at position E130 may be a substitution with any amino acid (X), i.e., E130 may be mutated to any amino acid except E (SEQ ID NO: 3). Preferably the mutation in amino acid position corresponding to E130 is a substitution with an aromatic or hydrophobic amino acid, more preferably the mutation in amino acid position E130 is a substitution with an aromatic or hydrophobic amino acid selected from the group consisting of Y, W, F, A, G, V, L, M and I. Thus, substitutions to amino acids not comprising polar, acidic or basic side chains are preferred. In certain embodiments the mutation is E130F, E130G or E130A, more preferably E130F or E130G (E130F/G) (SEQ ID NO: 4 or SEQ ID NO: 5, respectively). As used herein “X” refers to any amino acid, wherein the reference to a mutation as E130X specifies that X is any amino acid except for the original amino acid, i.e., E (Glu, glutamic acid). Without being bound by theory, substitution of the highly conserved glutamic acid (E) corresponding to amino acid position E130 in SEQ ID NO: 1 results in attenuation of the bacterial glutamine synthetase, wherein attenuation means reduced enzymatic activity in this context. Alternatively or in addition, the bacterial glutamine synthetase may have a point mutation in a highly conserved residue corresponding to position F226 of Providencia vermicola glutamine synthetase having the amino acid sequence of SEQ ID NO: 1. The point mutation corresponding to amino acid position F226 of SEQ ID NO: 1 is a substitution with the amino acid Y (Tyr, tyrosine), also referred to as F226Y. Without being bound by theory substituting highly conserved phenylalanine (F) corresponding to amino acid position F266 in SEQ ID NO: 1 with Y is believed to increase catalytic activity of glutamine synthetase. Such a mutation that increases catalytic activity of glutamine synthetase may be advantageously combined with a mutation that further attenuates the glutamine synthetase. In certain embodiments the mutation is E130X and/or F226Y, wherein X is any amino acid or as specified in this paragraph above. More preferably the mutation is E130X or E130X and F226Y. In a specific embodiment the mutation is E130F/G or E130F/G and F226Y. Alternatively or in addition, the bacterial glutamine synthetase may have a point mutation in a highly conserved residue corresponding to position R345 of Providencia vermicola glutamine synthetase having the amino acid sequence of SEQ ID NO: 1. The point mutation corresponding to amino acid position R345 of SEQ ID NO: 1 is a substitution with the amino acid A (Ala, alanine), also referred to as R345A or alternatively with another amino acid with aliphatic side chain, such as glycine (G), valine (V), leucine (L) or isoleucine (I), preferably glycine (G). Without being bound by theory substituting highly conserved arginine (R) corresponding to amino acid position R345 in SEQ ID NO: 1 results in attenuation of the bacterial glutamine synthetase, wherein attenuation means reduced enzymatic activity in this context. Optionally the bacterial glutamine synthetase may further comprise the point mutation F226Y. Thus, in certain embodiments the mutation is E130X and/or R345A (or G, V, L or I) optionally further comprising F226Y, wherein X is any amino acid or as specified in this paragraph above. Preferably the mutations is E130F/G and/or R345A, optionally further comprising F226Y, such as E130F/G and R345A, optionally further comprising F226Y, or preferably E130F/G or R345A, optionally further comprising F226Y.

In certain embodiments, the bacterial glutamine synthetase comprises an amino acid sequence having at least 85% sequence identity with the amino acid sequence of SEQ ID NO: 1, and a mutation selected from the group consisting of E130X, F226Y, R345A (or G, V, L or I) and combinations thereof, wherein X is any amino acid, preferably wherein the mutation in amino acid position E130 is a substitution with an aromatic and/or hydrophobic amino acid, more preferably selected from the group consisting of Y, W, F, A, G, V, L, M and I. In certain embodiments the mutation is E130X and/or F226Y, and wherein E130X is E130F, E130G or E130A, more preferably E130F or E130G (E130F/G). The term a “mutation of E130X, F226Y, R345A and combinations thereof” as used herein means at least one mutation of E130X and/or F226Y and/or R345A, i.e., (1) E130X or F226Y or R345A; or (2) E130X and F226Y, or R345A and F226Y, or E130X and R345A; or (3) E130X and F226Y and R345A. Preferably the mutation is (i) E130X or (ii) E130X and F226Y or (iii) R345A or (iv) R345A and F226Y, wherein X is as described herein.

In certain embodiments, the bacterial glutamine synthetase comprises an amino acid sequence having at least 95% sequence identity with the amino acid sequence of SEQ ID NO: 1 or SEQ ID NO: 14 and a mutation selected from the group consisting of E130X, F226Y, R345A (or G, V, L or I) and combinations thereof, wherein X is any amino acid, preferably wherein the mutation in amino acid position E130 is a substitution with an aromatic and/or hydrophobic amino acid, more preferably selected from the group consisting of Y, W, F, A, G, V, L, M and I. Preferably the amino acid sequence has at least 96%, preferably at least 98%, preferably at least 99% sequence identity with the amino acid sequence of SEQ ID NO: 1 or SEQ ID NO: 14, even more preferably the amino acid sequence has the amino acid sequence of SEQ ID NO: 1 or SEQ ID NO: 14, further comprising the mutation as disclosed herein, such as exemplified in the amino acid sequence of SEQ ID NOs: 3, 4, 5, 6, 7, 8, 9, 20, 21, 22 and 29 (based on SEQ ID NO: 1) or SEQ ID NOs: 23, 24, 25, 26, 27, 28 and 30 (based on SEQ ID NO: 14), preferably as in SEQ ID NOs: 3, 4, 5, 6, 7, 8, 9, 20 and 21 (based on SEQ ID NO: 1) or SEQ ID NOs: 23, 24, 25, 26 and 28 (based on SEQ ID NO: 14). In certain embodiments, the bacterial glutamine synthetase comprises an amino acid sequence having at least 95% sequence identity with the amino acid sequence of SEQ ID NO: 1, and a mutation selected from the group consisting of E130X, F226Y, R345A (or G, V, L or I) and combinations thereof, wherein X is any amino acid, preferably wherein the mutation in amino acid position E130 is a substitution with an aromatic and/or hydrophobic amino acid, more preferably selected from the group consisting of Y, W, F, A, G, V, L, M and I. In certain embodiments the mutation in the bacterial glutamine synthetase having at least 95% sequence identity with SEQ ID NO: 1 or SEQ ID NO: 14 is E130X and/or F226Y, and wherein E130X is E130F, E130G or E130A, more preferably E130F or E130G (E130F/G). In certain embodiments the mutation is E130X and/or F226Y and/or R345A, and wherein E130X is E130F, E130G or E130A, more preferably E130F or E130G (E130F/G). Thus, the bacterial glutamine synthetase may comprise a mutation (1) E130X or F226Y or R345A; or (2) E130X and F226Y, or R345A and F226Y, or E130X and R345A; or (3) E130X and F226Y and R345A. Preferably the mutation is E130X or R345A and optionally F226Y, i.e., (i) E130X or (ii) E130X and F226Y or (iii) R345A or (iv) R345A and F226Y. The term a “mutation E130X and/or F226Y” as used herein means a mutation E130X or F226Y or E130X and F226Y. More preferably the mutation is E130X or E130X and F226Y. The term a mutation “F226Y and/or R345A” as used herein means a mutation F226Y or R345A or F226Y and R345A, preferably R345A or F226Y and R345A.

The mammalian expression vector according to the invention is for expression of the heterologous sequence in mammalian cells, i.e., it is adapted for expression in mammalian cells (such as mammalian cells), i.e., expression of the bacterial glutamine synthetase and preferably further a protein of interest and/or a non-coding RNA. Thus, the mammalian expression vector according to the invention is characterized in that it comprises the polynucleotide encoding the bacterial glutamine synthetase operably linked to a mammalian promoter. Typically, the mammalian expression vector comprises an expression cassette comprising the polynucleotide encoding the bacterial glutamine synthetase operably linked to a mammalian promoter (such as a CMV promoter or an SV40 promoter). Mammalian promoters regulate transcription in mammalian cells, particularly mammalian promoter regulate transcription in mammalian cells. Exemplary mammalian promoters, without being limited thereto are simian virus 40 early promoter (SV40), cytomegalovirus immediate-early promoter (CMV), human ubiquitin C promoter (UBC), human elongation factor 1a promoter (EF1A), mouse phosphoglycerate kinase 1 promoter (PGK) and chicken β-actin promoter coupled to CMV early enhancer (CAGG). The mammalian expression vector may further comprise bacterial sequences, such as an origin of replication and resistance genes for vector amplification in bacterial cells.

The mammalian expression vector typically further comprises at least one polynucleotide encoding a protein of interest and/or a non-coding RNA, preferably an expression cassette comprising at least one polynucleotide encoding a protein of interest and/or a non-coding RNA. The polynucleotide encoding the protein of interest and/or the non-coding RNA is operably linked to a mammalian promoter. The protein of interest and/or the non-coding RNA and the bacterial glutamine synthetase may be encoded by the same (multicistronic) or separate expression cassettes. Preferably the protein of interest and/or non-coding RNA and the bacterial glutamine synthetase are encoded by at least two separate expression cassettes or at least two separately encoded mRNAs. The present invention does not encompass fusion proteins of the protein of interest with the bacterial glutamine synthetase or parts thereof. In certain embodiments, the non-coding RNA is an RNA interference (RNAi) mediating RNA, for example miRNA, siRNA, lncRNA or shRNA.

The term “expression cassette” as used herein is a distinct component of a DNA, particularly vector DNA, consisting of one or more coding polynucleotide sequences and the regulatory sequences controlling their expression in a transfected or transduced cell. An expression cassette comprises at least three components: a promoter sequence, an open reading frame, and termination sequence. In mammalian expression vectors comprising a polynucleotide encoding a protein of interest, the termination sequence is referred to as 3′ untranslated region and usually contains a polyadenylation site. The expression cassette directs the cell's machinery to make RNA and may therefore also be referred to as transcriptional cassette. The RNA may be coding RNA, further processed to mRNA encoding for a protein sequence e.g., glutamine synthetase or the protein of interest, or the RNA may be non-coding RNA, such as RNA interference (RNAi) mediating RNA, for example miRNA, siRNA, lncRNA or shRNA.

The protein of interest may be any protein, but is typically a therapeutic protein. The term “therapeutic protein” as used herein refers to proteins that can be used in medical treatment of humans and/or animals. These include, but are not limited to cytokines, growth factors, hormones, blood coagulation factors, vaccines, interferons, fusion proteins, antibodies, antibody-derived molecules and an antibody mimetics. In certain embodiments, the therapeutic protein is selected from the group consisting of a cytokine, a hormone, a fusion protein, an antibody, an antibody-derived molecule and an antibody mimetic.

In certain embodiments the protein of interest is an antibody. In cases where the protein of interest is an antibody, the mammalian expression vector comprises a polynucleotide comprising a coding sequence for a variable region of the heavy chain and/or a coding sequence for a variable region of the light chain of the antibody. In certain embodiments, the mammalian expression vector comprises a polynucleotide comprising a coding sequence for a heavy chain and/or a coding sequence for a light chain of the antibody. Thus, the polynucleotide comprising a coding sequence for a variable region of the heavy chain and the polynucleotide comprising a coding sequence of a variable region of the light chain may be expressed on the same mammalian expression vector or on separate mammalian expression vectors. The expression vector may comprise a multicistronic expression cassette, such as a bicistronic expression cassette, and/or multiple expression cassettes. A multicistronic expression cassette comprises more than one open reading frames separated by sequences coding for an RNA element that allows for translation initiation, such as an internal ribosomal entry site (IRES). In a multicistonic expression cassette, the two or more open reading frames are under the control of the same promoter. The polynucleotide encoding at least a variable region of the heavy chain and the polynucleotide encoding at least a variable region of the light chain may therefore be expressed within the same expression cassette (separated e.g., by an IRES sequence) or by two separate expression cassettes. Moreover, the bacterial glutamine synthetase and the protein of interest and/or the non-coding RNA may be expressed by the same or separate expression cassette(s). In case the protein of interest is an antibody, the bacterial glutamine synthetase and polynucleotide encoding at least a variable region of the heavy chain and/or the polynucleotide encoding at least a variable region of the light chain may be expressed by the same or separate expression cassettes or a mixture thereof.

In preferred embodiments, the mammalian expression vector is for stable integration into the host cell's genome (such as for stable transfection) and the integrating part of the vector comprises the polynucleotide encoding the bacterial glutamine synthetase and the at least one polynucleotide encoding the protein of interest and/or the non-coding RNA. The mammalian expression vector according to the invention, preferably the mammalian expression vector according to the invention, is a plasmid, a Bacterial Artificial Chromosome (BAC) or a viral vector. Said plasmid, a Bacterial Artificial Chromosome (BAC) or viral vector (e.g. a lentiviral vector) may be introduced into the mammalian host cell (such as the mammalian host cell) via transfection or transduction, respectively, and is preferably stably integrated into the host cell genome. The person skilled in the art knows suitable plasmids BACs or viral vectors and that a plasmid may further comprise transposon recognition sequences upstream and downstream of the polynucleotide encoding a bacterial synthetase as a selection marker and the optional at least one polynucleotide encoding a protein of interest and/or a non-coding RNA.

A preferred protein of interest is an antibody, including fragments and derivatives thereof. Typically, an antibody is monospecific, but an antibody may also be multispecific. Thus, the present invention may be used for the production of mono-specific antibodies, multi-specific antibodies, or fragments thereof, preferably of antibodies (mono-specific), bispecific antibodies, trispecific antibodies or fragments thereof, preferably antigen-binding fragments thereof. Exemplary antibodies within the scope of the present invention include but are not limited to anti-CD2, anti-CD3, anti-CD20, anti-CD22, anti-CD30, anti-CD33, anti-CD37, anti-CD40, anti-CD44, anti-CD44v6, anti-CD49d, anti-CD52, anti-EGFR1 (HER1), anti-EGFR2 (HER2), anti-GD3, anti-IGF, anti-VEGF, anti-TNFalpha, anti-IL2, anti-IL-5R or anti-IgE antibodies, and are preferably selected from the group consisting of anti-CD20, anti-CD33, anti-CD37, anti-CD40, anti-CD44, anti-CD52, anti-HER2/neu (erbB2), anti-EGFR, anti-IGF, anti-VEGF, anti-TNFalpha, anti-IL2 and anti-IgE antibodies.

The term “antibody”, “antibodies”, or “immunoglobulin(s)” is used herein in the broadest sense and encompasses various antibody structures, including but not limited to monoclonal antibodies, polyclonal antibodies, monospecific antibodies, multispecific antibodies (e.g., bispecific antibodies), and antibody fragments so long as they exhibit the desired antigen-binding activity. There are various classes of immunoglobulins: IgA, IgD, IgE, IgG, IgM, IgY, IgW. Preferably the antibody is an IgG antibody, more preferably an IgG1 or an IgG4 antibody.

Antibodies can be of any species and include chimeric and humanized antibodies. “Chimeric” antibodies are molecules in which antibody domains or regions are derived from different species. For example, the variable region of heavy and light chain can be derived from rat or mouse antibody and the constant regions from a human antibody. In “humanized” antibodies only minimal sequences are derived from a non-human species. Often only the CDR amino acid residues of a human antibody are replaced with the CDR amino acid residues of a non-human species such as mouse, rat, rabbit or llama. Sometimes a few key framework amino acid residues with impact on antigen binding specificity and affinity are also replaced by non-human amino acid residues.

Typically, antibodies are tetrameric polypeptides composed of two pairs of a heterodimer each formed by a heavy and a light chain. Stabilization of both the heterodimers as well as the tetrameric polypeptide structure occurs via interchain disulfide bridges. Each chain is composed of structural domains called “immunoglobulin domains” or “immunoglobulin regions” whereby the terms “domain” or “region” are used interchangeably. Each domain contains about 70-110 amino acids and forms a compact three-dimensional structure. Both heavy and light chain contain at their N-terminal end a “variable domain” or “variable region” with less conserved sequences which is responsible for antigen recognition and binding. The variable region of the light chain is also referred to as “VL” and the variable region of the heavy chain as “VH”.

An “antibody fragment” or “antigen-binding fragments” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments. Fab fragments consist of the variable regions of both chains, which are held together by the adjacent constant region. These may be formed by protease digestion, e.g., with papain, from conventional antibodies, but similarly Fab fragments may also be produced by genetic engineering. Further antibody fragments include F(ab′)2 fragments, which may be prepared by proteolytic cleavage with pepsin.

Using genetic engineering methods it is possible to produce shortened antibody fragments which consist only of the variable regions of the heavy (VH) and of the light chain (VL). These are referred to as Fv fragments (Fragment variable=fragment of the variable part). Since these Fv-fragments lack the covalent bonding of the two chains by the cysteines of the constant chains, the Fv fragments are often stabilized. It is advantageous to link the variable regions of the heavy and of the light chain by a short peptide fragment, e.g. of 10 to 30 amino acids, preferably 15 amino acids. In this way a single peptide strand is obtained consisting of VH and VL, linked by a peptide linker. An antibody protein of this kind is known as a single-chain-Fv (scFv). Examples of scFv-antibody proteins are known to the person skilled in the art. Thus, antibody fragments and antigen-binding fragments further include Fv-fragments and particularly scFv.

In recent years, various strategies have been developed for preparing scFv as a multimeric derivative. This is intended to lead, in particular, to recombinant antibodies with improved pharmacokinetic and biodistribution properties as well as with increased binding avidity. In order to achieve multimerisation of the scFv, scFv were prepared as fusion proteins with multimerisation domains. The multimerisation domains may be, e.g. the CH3 region of an IgG or coiled coil structure (helix structures) such as Leucine-zipper domains. However, there are also strategies in which the interaction between the VH/VL regions of the scFv is used for the multimerisation (e.g. dia-, tri- and pentabodies). By diabody the skilled person means a bivalent homodimeric scFv derivative. The shortening of the linker in a scFv molecule to 5-10 amino acids leads to the formation of homodimers in which an inter-chain VH/VL-superimposition takes place. Diabodies may additionally be stabilized by the incorporation of disulfide bridges. Examples of diabody-antibody proteins are known from the prior art.

By minibody the skilled person means a bivalent, homodimeric scFv derivative. It consists of a fusion protein which contains the CH3 region of an immunoglobulin, preferably IgG, most preferably IgG1 as the dimerisation region which is connected to the scFv via a Hinge region (e.g. also from IgG1) and a linker region. Examples of minibody-antibody proteins are known from the prior art.

By triabody the skilled person means a: trivalent homotrimeric scFv derivative. ScFv derivatives wherein VH-VL is fused directly without a linker sequence lead to the formation of trimers.

The skilled person will also be familiar with so-called miniantibodies which have a bi-, tri- or tetravalent structure and are derived from scFv. The multimerisation is carried out by di-, tri- or tetrameric coiled coil structures. In a preferred embodiment of the present invention, the gene of interest is encoded for any of those desired polypeptides mentioned above, preferably for a monoclonal antibody, a derivative or fragment thereof.

Further encompassed is a single-domain antibody (sdAb), also be referred to as nanobody, which is an antibody fragment of a single monomeric variable antibody domain. Single-domain antibodies are typically engineered from heavy chain antibodies found in camelids (V_{H}H fragments) or cartilaginous fishes (V_{NAR} fragments).

The immunoglobulin fragments composed of the CH2 and CH3 domains of the antibody heavy chain are called “Fc fragments”, “Fc region” or “Fc” because of their crystallization propensity (Fc=fragment crystallizable). These may be formed by protease digestion, e.g. with papain or pepsin from conventional antibodies but may also be produced by genetic engineering. The N-terminal part of the Fc fragment might vary depending on how many amino acids of the hinge region are still present.

Antibodies comprising an antigen-binding fragment and an Fc region may also be referred to as full-length antibody. Full-length antibody may be mono-specific and multispecific antibodies. Multispecific antibodies are antibodies which have at least two different antigen-binding sites each of which bind to different epitopes. A multispecific antibody includes bispecific and trispecific antibodies. A bispecific antibody has two different binding binding sites. Multispecific antibodies also include antibody formats other than full-length antibodies such as antibody-derived molecules.

Bispecific antibodies typically combine antigen-binding specificities for target cells (e.g., malignant B cells) and effector cells (e.g., T cells, NK cells or macrophages) in one molecule. Exemplary bispecific antibodies, without being limited thereto are diabodies, BiTE (Bi-specific T-cell Engager) formats and DART (Dual-Affinity Re-Targeting) formats. The diabody format separates cognate variable domains of heavy and light chains of the two antigen binding specificities on two separate polypeptide chains, with the two polypeptide chains being associated non-covalently. The DART format is based on the diabody format, but it provides additional stabilization through a C-terminal disulfide bridge. Trispecific antibodies are monoclonal antibodies which combine three antigen-binding specificities. They may be build on bispecific-antibody technology that reconfigures the antigen-recognition domain of two different antibodies into one bispecific molecule. For example, trispecific antibodies have been generated that target CD38 on cancer cells and CD3 and CD28 on T cells. Multispecific antibodies are particularly difficult to product with high product quality.

The term “antibody-derived molecule” as used herein refers to any molecule comprising at least an antigen-binding moiety that is structurally related to antibodies. It includes modified full-length mono- or bispecific antibodies further modified with an additional antigen binding moiety or smaller antibody formats including the ones described herein.

The term “antibody mimetic” as used herein refers to proteins that bind to specific antigens in a manner similar to antibodies, but that are not structurally related to antibodies. Antibody mimetic include, without being limited thereto an anticalin, an affibody, an adnectin, a monobody, a DARPin, an affimer, and an affitin.

A single-domain antibody (sdAb) may also be referred to as nanobody. The person skilled in the art will understand that the protein may comprise more than one antigen-binding domain and hence may be multivalent, preferably bivalent (e.g., a bivalent sdAb or a bivalent anticalin or any other bivalent antibody mimetic).

Another preferred therapeutic protein is a fusion protein, such as an Fc-fusion protein. Thus, the invention can be advantageously used for production of fusion proteins, such as Fc-fusion proteins. The effector part of the fusion protein can be the complete sequence or any part of the sequence of a natural or modified heterologous protein. The immunoglobulin constant domain sequences may be obtained from any immunoglobulin subtypes, such as IgG1, IgG2, IgG3, IgG4, IgA1 or IgA2 subtypes or classes such as IgA, IgE, IgD or IgM. Preferentially they are derived from human immunoglobulin, more preferred from human IgG and even more preferred from human IgG1 and IgG2. Non-limiting examples of Fc-fusion proteins are MCP1-Fc, ICAM-Fc, EPO-Fc and scFv fragments or the like coupled to the CH2 domain of the heavy chain immunoglobulin constant region comprising the N-linked glycosylation site. Fc-fusion proteins can be constructed by genetic engineering approaches by introducing the CH2 domain of the heavy chain immunoglobulin constant region comprising the N-linked glycosylation site into another expression construct comprising for example other immunoglobulin domains, enzymatically active protein portions, or effector domains. Thus, an Fc-fusion protein according to the present invention comprises also a single chain Fv fragment linked to the CH2 domain of the heavy chain immunoglobulin constant region comprising, e.g., the N-linked glycosylation site.

The term “cytokine” refers to small proteins, which are released by cells and act as intercellular mediators, for example influencing the behavior of the cells surrounding the secreting cell. Cytokines may be secreted by immune cells or other cells, such as T-cells, B-cells, NK cells and macrophages. Cytokines may be involved in intercellular signaling events, such as autocrine signaling, paracrine signaling and endocrine signaling. They may mediate a range of biological processes including, but not limited to immunity, inflammation, and hematopoiesis. Cytokines may be chemokines, interferons, interleukins, lymphokines or tumor necrosis factors.

As used herein, “growth factor” refers to proteins or polypeptides that are capable of stimulating cell growth.

In a related aspect the invention further relates to a use of the mammalian expression vector of the invention for expression of a protein of interest and/or a non-coding RNA in a mammalian cell, particularly a rodent cell, such as a CHO cell. The bacterial glutamine synthetase encoded by said mammalian expression vector serves as a selection marker in said mammalian cells.

In a second aspect, the invention relates to a nucleic acid sequence comprising a polynucleotide encoding a bacterial glutamine synthetase comprising an amino acid sequence having at least 85% sequence identity with the amino acid sequence of SEQ ID NO: 1 operably linked to a mammalian promoter, optionally further comprising at least one polynucleotide encoding a protein of interest and/or a non-coding RNA. The nucleic acid sequence may be part of the mammalian expression vector of the first aspect. Thus, the embodiments and examples specified with regard to the first aspect similarly apply to this aspect. In particular, in certain embodiments the bacterial glutamine synthetase comprises an amino acid sequence having at least 85%, at least 90% or at least 95% sequence identity with the amino acid sequence of SEQ ID NO: 1, and a mutation selected from the group consisting of E130X, F226Y, R345A and combinations thereof (such as E130X and/or F226Y), wherein X is any amino acid, preferably wherein the mutation in amino acid position E130 is a substitution with an aromatic or hydrophobic amino acid, more preferably selected from the group consisting of Y, W, F, A, G, V, L, M and I. Preferably, the mutation is E130X and/or F226 and E130X is E130F or E130G; or the mutation is F226Y and/or R345A; or the mutation is E130X and/or R345A, optionally further comprising the mutation F226Y.

In yet a third aspect, the invention relates to a bacterial glutamine synthetase comprising an amino acid sequence having at least 85% sequence identity with the amino acid sequence of SEQ ID NO: 1 and a mutation selected from the group consisting of E130X, F226Y, R345A and combinations thereof, wherein X is any amino acid, preferably wherein the mutation in amino acid position E130 is a substitution with an aromatic or hydrophobic amino acid, more preferably selected from the group consisting of Y, W, F, A, G, V, L, M and I. In certain embodiments the invention relates to a bacterial glutamine synthetase, such as from Providencia vermicola, comprising an amino acid sequence having at least 95% sequence identity with the amino acid sequence of SEQ ID NO: 1 and a mutation selected from the group consisting of E130X, F226Y, R345A and combinations thereof (such as E130X and/or F226Y), wherein X is any amino acid, preferably wherein the mutation in amino acid position E130 is a substitution with an aromatic or hydrophobic amino acid, more preferably selected from the group consisting of Y, W, F, A, G, V, L, M and I; or to a bacterial glutamine synthetase comprising an amino acid sequence having at least 95% sequence identity with the amino acid sequence of SEQ ID NO: 1 or SEQ ID NO: 14 and a mutation E130X and/or F226Y and/or R345A, wherein X is any amino acid, preferably wherein the mutation in amino acid position E130 is a substitution with an aromatic or hydrophobic amino acid, more preferably selected from the group consisting of Y, W, F, A, G, V, L, M and I. Preferably, the mutation is E130X and/or F226 and E130X is E130F or E130G; or the mutation is F226Y and/or R345A; or the mutation is E130X and/or R345A optionally further comprising the mutation F226Y, preferably E130X or R345A optionally further comprising the mutation F226Y. The embodiments and examples specified with regard to the first and second aspect similarly apply to this aspect.

In a fourth aspect, the invention relates to a kit comprising the expression vector according to the invention and optionally a cell culture medium not comprising glutamine.

In a fifth aspect, the invention relates to a use of a bacterial glutamine synthetase as a selection marker in mammalian cells. The bacterial glutamine synthetase is further specified as disclosed with regard to the first aspect above. More specifically, bacterial glutamine synthetase is derived from bacteria of the order Enterobacterales and of the family Monganellaceae, preferably of a Providencia species or a Photorhabdus species, more preferably of Providencia vermicola or Photorhabdus luminescens, even more preferably of Providencia vermicola. Preferably, the bacterial glutamine synthetase comprises an amino acid sequence having at least 85% sequence identity with the amino acid sequence of SEQ ID NO: 1. In certain embodiments the bacterial glutamine synthetase comprises an amino acid sequence having at least 85%, at least 90% and preferably at least 95% sequence identity with SEQ ID NO: 1. In other embodiment, the bacterial glutamine synthetase comprises an amino acid sequence having at least 95%, at least 96%, at least 97%, at least 98%, at least 99% or 100% sequence identity with the amino acid sequence of SEQ ID NO: 1 or at least 95%, at least 96%, at least 97%, at least 98%, at least 99% or 100% sequence identity with the amino acid sequence of SEQ ID NO: 14. Upon transfection or transduction of a mammalian expression vector comprising said bacterial glutamine synthetase, the bacterial glutamine synthetase mediates increased selection stringency and/or genetic stability of a stably co-integrated protein of interest and/or non-coding RNA in CHO cells compared to a CHO glutamine synthetase having the amino acid sequence of SEQ ID NO: 2. In preferred embodiments, the bacterial glutamine synthetase is derived from a Providencia species and/or comprises an amino acid sequence having at least 95% sequence identity with amino acid sequence of SEQ ID NO: 1; more preferably the bacterial glutamine synthetase is a Providencia vermicola glutamine synthetase. In another preferred embodiments, the bacterial glutamine synthetase is derived from a Photorhabdus species and/or comprises an amino acid sequence having at least 95% sequence identity with amino acid sequence of SEQ ID NO: 14; more preferably the bacterial glutamine synthetase is a Photorhabdus luminescens glutamine synthetase. The amino acid sequence of Providencia vermicola glutamine synthetase (SEQ ID NO: 1) and Photorhabdus luminescens glutamine synthetase (SEQ ID NO: 14) have a sequence identity of 87.2% (see Table B).

The bacterial glutamine synthetase may further comprise point mutations that further attenuate the glutamine synthetase (e.g., E130X and/or R345A) or that increase catalytic activity (e.g., F226Y). Particularly, the bacterial glutamine synthetase may have a point mutation in a highly conserved residue corresponding to position E130 of Providencia vermicola glutamine synthetase having the amino acid sequence of SEQ ID NO: 1. The point mutation at position E130 may be a substitution with any amino acid (X), preferably the mutation in amino acid position corresponding to E130 is a substitution with an aromatic or hydrophobic amino acid, such as a substitution with an aromatic or hydrophobic amino acid selected from the group consisting of Y, W, F, A, G, V, L, M and I. In certain embodiments the mutation is E130F, E130G or E130A, more preferably E130F or E130G (E130F/G) (SEQ ID NO: 4 or SEQ ID NO: 5, respectively). Alternatively or in addition, the bacterial glutamine synthetase may have a point mutation in a highly conserved residue corresponding to position F226 of Providencia vermicola glutamine synthetase having the amino acid sequence of SEQ ID NO: 1, wherein the point mutation is a substitution with the amino acid Y (Tyr, tyrosine), also referred to as F226Y. In certain embodiments the mutation is E130X and/or F226Y, wherein X is any amino acid or as specified in this paragraph above. More preferably the mutation is E130X or E130X and F226Y. In a specific embodiment the mutation is E130F/G or E130F/G and F226Y. Alternatively or in addition, the bacterial glutamine synthetase may have a point mutation in a highly conserved residue corresponding to position R345 of Providencia vermicola glutamine synthetase having the amino acid sequence of SEQ ID NO: 1. The point mutation corresponding to amino acid position R345 of SEQ ID NO: 1 is a substitution with the amino acid A (Ala, alanine), also referred to as R345A or alternatively with another amino acid with aliphatic side chain, such as glycine (G), valine (V), leucine (L) or isoleucine (I), preferably glycine (G). Without being bound by theory substituting highly conserved arginine (R) corresponding to amino acid position R345 in SEQ ID NO: 1 results in attenuation of the bacterial glutamine synthetase, wherein attenuation means reduced enzymatic activity in this context. Optionally the bacterial glutamine synthetase may further comprise the point mutation F226Y. Thus, in certain embodiments the mutation is E130X and/or R345A (or G, V, L or I) optionally further comprising F226Y, wherein X is any amino acid or as specified in this paragraph above. Preferably the mutations is E130F/G and/or R345A, optionally further comprising F226Y, such as E130F/G and R345A, optionally further comprising F226Y, or preferably E130F/G or R345A, optionally further comprising F226Y. Preferably the bacterial glutamine synthetase comprises an amino acid having at least 85% sequence identity with the amino acid sequence of SEQ ID NO: 1, further comprising the point mutations as indicated above.

In preferred embodiments, the bacterial glutamine synthetase comprises an amino acid sequence having at least 95% sequence identity with the amino acid sequence of SEQ ID NO: 1 or SEQ ID NO: 14, and a mutation selected from the group consisting of E130X, F226Y, R345A (or G, V, L or I) and combinations thereof (e.g. E130X and/or F226Y), wherein X is any amino acid, preferably wherein the mutation in amino acid position E130 is a substitution with an aromatic and/or hydrophobic amino acid, more preferably selected from the group consisting of Y, W, F, A, G, V, L, M and I. Preferably the amino acid sequence has at least 96%, preferably at least 98%, preferably at least 99% sequence identity with the amino acid sequence of SEQ ID NO: 1 or SEQ ID NO: 14, even more preferably the amino acid sequence has the amino acid sequence of SEQ ID NO: 1 or SEQ ID NO: 14, further comprising the mutation as disclosed herein, such as exemplified in the amino acid sequence of SEQ ID NOs: 3, 4, 5, 6, 7, 8, 9, 20, 21, 22 and 29 (based on SEQ ID NO: 1) or SEQ ID NOs: 23, 24, 25, 26, 27, 28 and 30 (based on SEQ ID NO: 14), preferably SEQ ID NOs: 3, 4, 5, 6, 7, 8, 9, 20 and 21 (based on SEQ ID NO: 1) or SEQ ID NOs: 23, 24, 25, 26 and 28 (based on SEQ ID NO: 14). In certain embodiments the mutation is E130X and/or F226Y, and preferably wherein E130X is E130F, E130G or E130A, more preferably E130F or E130G (E130F/G). In certain embodiments the mutation is F226Y and/or R345A, more preferably R345A or R345A and F226Y. In certain embodiments the mutation is E130X and/or R345A (or G, V, L or I) optionally further comprising F226Y, wherein X is any amino acid or as specified in this paragraph above. Preferably the mutations is E130F/G and/or R345A, optionally further comprising F226Y, such as E130F/G and R345A, optionally further comprising F226Y, or preferably E130F/G or R345A, optionally further comprising F226Y.

In a sixth aspect, the invention relates to a mammalian host cell comprising the mammalian expression vector of the invention (first aspect), the nucleic acid sequence of the invention (second aspect) or a nucleic acid sequence encoding the bacterial glutamine synthetase of the invention (third aspect). The mammalian host cell may be any host cell, provided the host cell is an immortalized cell and not a primary cell. In certain embodiments, the mammalian host cell is a mouse, a human or a rodent cell, more preferably a rodent cell, even more preferably a CHO cell. Moreover, the mammalian host cell is preferably a GS gene knockout cell (GS knockout mutant) host cell. The term “GS gene knockout cell” as used herein refers to a cell in which the endogenous GS gene has been knocked out, i.e., deleted or disrupted, resulting in GS enzyme function disruption. Such cells may be referred to as GS−/− or GS−/+ cells, depending on whether both or only one allele has been deleted or disrupted. Extracellular glutamine supplementation or a GS gene introduced by an expression vector is essential for cell survival of GS gene knockout cells. In a preferred embodiment the mammalian host cell is a CHO-K1 cell, more preferably a CHO-K1-GS (GS−/−) cell. In certain embodiments the mammalian host cell is a monoclonal cell line generated by a step of single cell cloning and clonal expansion. Preferably, the mammalian host cell comprises a polynucleotide encoding a bacterial glutamine synthetase and at least one polynucleotide encoding a protein of interest and/or the non-coding RNA co-integrated into the host cell genome.

### Method for Preparing a Cell Line or Producing a Protein

In a seventh aspect, the invention relates to a method for preparing a cell stably expressing a protein of interest and/or a non-coding RNA, comprising (a) introducing the expression vector according to the invention (first aspect) or the nucleic acid of the invention (second aspect) comprising a polynucleotide encoding a bacterial glutamine synthetase and at least one polynucleotide encoding a protein of interest and/or a non-coding RNA into a mammalian host cell; and (b) culturing the mammalian host cell in a medium comprising no glutamine under conditions to select for the bacterial glutamine synthetase, wherein the at least one polynucleotide encoding a protein of interest and/or a non-coding RNA is co-integrated with the polynucleotide encoding the bacterial glutamine synthetase into the host cell genome. Optionally the method may further comprise a step of culturing the resulting cell pool stably expressing a protein of interest and/or a non-coding RNA selected in step (b) and/or isolating a cell pool stably expressing a protein of interest and/or a non-coding RNA. A cell stably expressing a protein of interest and/or a non-coding RNA means that the polynucleotide encoding the protein or interest and/or the non-coding RNA is stable integrated into the genome of the host cell and that the protein of interest and/or the non-coding RNA is stable expressed, i.e., over an extended period of time, such as at least 40 days preferably for months or years.

The mammalian expression vector or the nucleic acid may be a plasmid, a Bacterial Artificial Chromosome (BAC) or a viral vector. Thus, the mammalian expression vector or the nucleic acid of the invention may be introduced by transfection or transduction, respectively. Preferably the mammalian expression vector or the nucleic acid is a plasmid. More preferably the mammalian expression vector or the nucleic acid is introduced by stable transfection. Methods of transducing or transfecting a mammalian expression vector or a nucleic acid into mammalian cells are well known in the art and comprise chemical means, such as calcium phosphate precipitation and lipofection, and physical means, such as electroporation. The polynucleotide encoding the bacterial glutamine synthetase and the polynucleotide encoding the protein of interest and/or the non-coding RNA are operably linked to a mammalian promoter and are therefore adapted for expression in a mammalian host cell.

The method of the invention may further comprise a step (c) of isolating a single clone for clonal expansion to prepare a monoclonal cell line. The person skilled in the art would understand that transfection or transduction often requires a large number of cells, resulting in a heterogenous pool of recombinant cells with, e.g., varying integration site populations. For generating a clonal cell line, the cell pool is diluted or sorted for single cell isolation (monoclonal) and each single clone is subjected to clonal expansion to prepare a monoclonal cell line. The term “cell line” as used herein refers to a population of cell derived from a single cell clone and can be grown for an unlimited time. It is therefore also referred to as monoclonal cell line. Thus, a cell line is genetically stable and hence the characteristics of a cell line should not change over time. Particularly phenotypic characteristics such as production levels (titer) and growth rate and density (VCD and maximal VCD), viability as well as genetic integrity as measured via copy number and DNA-fingerprint assays should be maintained when cultured under comparable conditions.

The cell pool or the monoclonal cell line prepared according to the method of the invention may be further used for stably producing a protein of interest or for stably producing a non-coding RNA, such as an RNA mediating RNAi, e.g., an miRNA, an siRNA, lncRNA or an shRNA. RNAi is used for gene silencing and may therefore be used for generating a cell pool or a monoclonal cell line in beneficial properties for, e.g., protein production. For example, a difficult to remove host cell protein (HCP) may be silenced in the cell pool or monoclonal cell line, or an enzyme such as a fucosyltransferase may be silenced to modify the glycosylation profile of the cell pool or monoclonal cell line. Moreover, CHO cells commonly used for large-scale industrial production are often engineered to improve their characteristics in the production process, or to facilitate selection of recombinant cells. Such engineering includes, but is not limited to increasing apoptosis resistance, reducing autophagy, increasing cell proliferation, altered expression of cell-cycle regulating proteins, chaperone engineering, engineering of the unfolded protein response (UPR), engineering of secretion pathways and metabolic engineering. Such engineering can potentially be achieved using RNAi in mammalian host cells generated by the methods of the present invention.

In a related eighth aspect, the invention relates to a method of producing a protein of interest, comprising (a) introducing the mammalian expression vector according the invention or the nucleic acid according to the invention into a mammalian host cell, wherein the expression vector comprises a polynucleotide encoding a bacterial glutamine synthetase operably linked to a mammalian promoter and at least one polynucleotide encoding a protein of interest; (b) culturing the mammalian host cell in a medium comprising no glutamine under conditions to select for the bacterial glutamine synthetase, wherein the at least one polynucleotide encoding a protein of interest is co-integrated with the polynucleotide encoding the bacterial glutamine synthetase into the host cell genome; (c) optionally isolating a single clone for clonal expansion to prepare a monoclonal cell line; (d) culturing the mammalian host cell under conditions to produce the protein of interest; and (e) harvesting and optionally purifying the protein of interest. The mammalian expression vector or the nucleic acid of the invention may be introduced by transfection or transduction. Preferably the mammalian expression vector or the nucleic acid is introduced by stable transfection.

The person skilled in the art would understand that for a mammalian expression vector typically only a part of the vector is integrated into the host cell's genome. Thus, the integrating part of the vector comprises the polynucleotide encoding the bacterial glutamine synthetase and at least one polynucleotide encoding the protein of interest and/or the non-coding RNA. Moreover, the integrated part of the mammalian expression vector or the integrated nucleic acid sequence of the invention may further be amplified, e.g., by increasing the concentration of a glutamine synthetase inhibitor, such as methionine sulfoximine (MSX). Amplification is optional and may result in higher productivity due to higher copy numbers of the polynucleotide encoding the protein of interest and/or the non-coding RNA, because these become co-amplified together with the bacterial glutamine synthetase. The methods according to the invention may include the generation of a cell pool or a monoclonal cell line. Further, a mammalian host cell generated according to the methods of the invention or a mammalian host cell according to the invention may further be used for producing a protein of interest and/or a non-coding RNA or in a method for producing a protein of interest.

Thus, in a further nineth aspect, the invention relates to a method of producing a protein of interest, comprising (a) providing the mammalian host cell according to the invention or produced by the methods according to the invention comprising a polynucleotide encoding a bacterial glutamine synthetase operably linked to a mammalian promoter and at least one polynucleotide encoding a protein of interest; (b) culturing the mammalian host cell under conditions to produce the protein of interest; and (c) harvesting and optionally purifying the protein of interest.

In preferred embodiments of the methods of the invention, the mammalian host cell is a GS gene knockout cell. The person skilled in the art will understand that this refers to the endogenous GS gene, while the bacterial GS gene is present in the host cell following transfection or transduction of the mammalian expression vector or the nucleic acid sequence of the invention. The mammalian host cell (transfected or transduced with the mammalian expression vector or the nucleic acid sequence of the invention) is cultured in a medium comprising no glutamine under conditions to select for the bacterial glutamine synthetase in step (b). This may further comprise the addition of the GS inhibitor methionine sulfoximine (MSX).

In certain embodiments of the methods of the invention the cell (cell pool) or monoclonal cell line is generated with increased selection stringency and/or has increased genetic stability and/or has higher productivity compared to a cell or cell line generated with a glutamine synthetase from Cricetulus griseus having the amino acid sequence of SEQ ID NO: 2. Genetic stability may be quantified by measuring copy numbers of the integrated transgenes. Genetic stability is assessed by measuring copy number over an extended cultivation time. In addition to that genomic rearrangements are monitored via e.g. Southern Blot analysis. An increase in selection stringency may be determined by the duration until reaching >70% viability.

The mammalian host cell may be any host cell, provided the host cell is an immortalized cell and not a primary cell. The methods described herein are in vitro methods and the mammalian host cells according to the invention are for in vitro use in cell culture. The term “mammalian cell” as used herein refers to mammalian cell lines suitable for the production of a product of interest, such as a heterologous protein of interest and/or a non-coding RNA and may also be referred to as “host cells” or “mammalian host cell”. The mammalian cells are preferably transformed and/or immortalized cell lines. They are adapted to serial passages in cell culture, preferably serum-free cell culture and/or preferably as suspension culture, and do not include primary non-transformed cells or cells that are part of an organ structure.

Preferably the mammalian host cell is a mouse, a human or rodent cell, more preferably a rodent cell, even more preferably a CHO cell. Preferred mammalian cells for heterologous protein production are murine cells, rodent cells or human cells. Preferred examples of mammalian cells or mammalian cell lines are CHO cells (such as DG44 and K1), NS0 cells, HEK293 cells (such as HEK293 cells and HEK293T cells) and BHK21 cells. Preferably the mammalian cells or mammalian cell lines are adapted to growth in suspension. In a preferred embodiment the mammalian cells or mammalian cell line is a CHO cell. In certain embodiments the mammalian cell is a HEK293 cell or a CHO cell or a HEK293 cell or a CHO cell derived cell, preferably the mammalian cell is a CHO cell or a CHO derived cell.

Suitable rodent cells may be e.g., hamster cells, particularly BHK21, BHK TK, CHO, CHO-K1, CHO-DXB11 (also referred to as CHO-DUKX or DuxB11), a CHO-S cell and CHO-DG44 cells or the derivatives/progenies of any of such cell line. Particularly preferred are CHO cells, such as CHO-DG44, CHO-K1 and BHK21, and even more preferred are CHO-DG44 and CHO-K1 cells. Most preferred are CHO-DG44 cells. Glutamine synthetase (GS)-deficient derivatives of the mammalian cell, particularly of the CHO-DG44 and CHO-K1 cell are also encompassed. In one embodiment of the invention the mammalian cell is a Chinese hamster ovary (CHO) cell, preferably a CHO-DG44 cell, a CHO-K1 cell, a CHO DXB11 cell, a CHO-S cell, a CHO GS deficient cell or a derivative thereof. Suitable human cells are HEK293 or HEK293T cells. The host cells may also be murine cells such as murine myeloma cells, such as NS0 and Sp2/0 cells or the derivatives/progenies of any of such cell line.

Moreover, the mammalian host cell is preferably a GS gene knockout cell (GS knockout mutant). The term “GS gene knockout cell” as used herein refers to a cell in which the endogenous GS gene has been knocked out, i.e., deleted or disrupted, resulting in GS enzyme function disruption. Such cells may be referred to as GS−/− or GS−/+ cells, depending on whether both or only one allele has been deleted or disrupted. Extracellular glutamine supplementation or a GS gene introduced by an expression vector is essential for cell survival of GS gene knockout cells. In a preferred embodiment the mammalian host cell is a CHO-K1 cell, more preferably a CHO-K1-GS (GS−/−) cell.

Preferably, CHO cells that allow for efficient cell line development processes are metabolically engineered, such as by endogenous glutamine synthetase (GS) knockout to facilitate selection with methionine sulfoximine (MSX).

Non-limiting examples of mammalian cells which can be used in the meaning of this invention are also summarized in Table A. However, derivatives/progenies of those cells, other mammalian cells, including but not limited to human, mice, rat, monkey, and rodent cell lines, can also be used in the present invention, particularly for the production of biopharmaceutical proteins.

TABLE A
Exemplary mammalian production cell lines

| Cell line | Order Number |
|---|---|
| NS0 | ECACC No. 85110503 |
| Sp2/0-Ag14 | ATCC CRL-1581 |
| BHK21 | ATCC CCL-10 |
| BHK TK⁻ | ECACC No. 85011423 |
| HaK | ATCC CCL-15 |
| 2254-62.2 (BHK-21 derivative) | ATCC CRL-8544 |
| CHO | ECACC No. 8505302 |
| CHO wild type | ECACC 00102307 |
| CHO-K1 | ATCC CCL-61 |
| CHOZN ® | Merck SAFC |
| | GS−/− and DHFR−/− |
| CHO-DUKX | ATCC CRL-9096 |
| (=CHO duk⁻, CHO/dhfr, CHO-DXB11) | |
| CHO-DUKX 5A-HS-MYC | ATCC CRL-9010 |
| CHO-DG44 | Urlaub G, et al., 1983. Cell. 33: 405-412. |
| CHO Pro-5 | ATCC CRL-1781 |
| CHO-S | Life Technologies A1136401; CHO-S is derived |
| | from CHO as disclosed in Deaven LL and Petersen |
| | DF, 1973, Chromosoma (Berl.) 41: 129-144; and |
| | Lewis NE et al., 2013, Nature Biotechnology 31: |
| | 759-765 |
| V79 | ATCC CCC-93 |
| B14AF28-G3 | ATCC CCL-14 |
| HEK 293 | ATCC CRL-1573 |
| COS-7 | ATCC CRL-1651 |
| U266 | ATCC TIB-196 |
| HuNS1 | ATCC CRL-8644 |
| CHL | ECACC No. 87111906 |
| CAP¹ | Wölfel J, et al., 2011. BMC Proc. 5(Suppl 8): P133. |
| PER.C6 ® | Pau et al., 2001. Vaccines. 19: 2716-2721. |
| H4-II-E | ATCC CRL-1548 |
| | ECACC No. 87031301 |
| | Reuber, 1961. J. Natl. Cancer Inst. 26: 891-899. |
| | Pitot HC, et al., 1964. Natl. Cancer Inst. Monogr. |
| | 13: 229-245 |
| H4-II-E-C3 | ATCC CRL-1600 |
| H4TG | ATCC CRL-1578 |
| H4-II-E | DSM ACC3129 |
| H4-II-Es | DSM ACC3130 |

¹CAP (CEVEC's Amniocyte Production) cells are an immortalized cell line based on primary human amniocytes. They were generated by transfection of these primary cells with a vector containing the functions E1 and pIX of adenovirus 5. CAP cells allow for competitive stable production of recombinant proteins with excellent biologic activity and therapeutic efficacy as a result of authentic human posttranslational modification.

Cells are most preferred, when being established, adapted, and completely cultivated under serum free conditions, and optionally in media, which are free of any protein/peptide of animal origin. Commercially available media such as Ham's F12 (Sigma, Deisenhofen, Germany), RPMI-1640 (Sigma), Dulbecco's Modified Eagle's Medium (DMEM; Sigma), Minimal Essential Medium (MEM; Sigma), Iscove's Modified Dulbecco's Medium (IMDM; Sigma), CD-CHO (Invitrogen, Carlsbad, CA), serum-free CHO Medium (Sigma), and protein-free CHO Medium (Sigma) are exemplary appropriate nutrient solutions. Any of the media may be supplemented as necessary with a variety of compounds, non-limiting examples of which are recombinant hormones and/or other recombinant growth factors (such as insulin, transferrin, epidermal growth factor, insulin like growth factor), salts (such as sodium chloride, calcium, magnesium, phosphate), buffers (such as HEPES), nucleosides (such as adenosine, thymidine), glutamine, glucose or other equivalent energy sources, antibiotics and trace elements. Any other necessary supplements may also be included at appropriate concentrations that would be known to those skilled in the art. For the growth and selection of genetically modified cells expressing a selectable gene a suitable selection agent is added to the culture medium.

The protein of interest encoded by the mammalian expression vector or produced by the methods of the invention is preferably produced in CHO cells in cell culture. Following expression, the recombinant protein is harvested and further purified. The antibody may be recovered from the culture medium as a secreted protein in the harvested cell culture fluid (HCCF) or from a cell lysate (i.e., the fluid containing the content of a cell lysed by any means, including without being limited thereto enzymatic, chemical, osmotic, mechanical and/or physical disruption of the cell membrane and optionally cell wall) and purified using techniques described herein. According to the invention the method comprises providing a harvested cell culture fluid comprising a protein of interest, such as an antibody as starting material, wherein the HCCF is from CHO cell culture. Preferably the protein of interest, such as the antibody, is recovered from the harvested cell culture fluid following cell separation, such as by filtration and/or centrifugation. Thus, in certain embodiments the harvest includes centrifugation and/or filtration to produce a harvested cell culture fluid.

In view of the above, it will be appreciated that the invention also encompasses the following items:
Item 1 provides a mammalian expression vector comprising a polynucleotide encoding a bacterial glutamine synthetase as a selection marker (such as a mammalian expression vector), wherein the bacterial glutamine synthetase comprises an amino acid sequence having at least 85% sequence identity with the amino acid sequence of SEQ ID NO: 1.
Item 2 specifies for the mammalian expression vector of item 1 that the bacterial glutamine synthetase is derived from bacteria of the order Enterobacterales and of the family of Morganellaceae.
Item 3 specifies for the mammalian expression vector of item 1 or 2 that the bacterial glutamine synthetase is derived from a Providencia species or a Photorhabdus species, preferably wherein the bacterial glutamine synthetase is a Providencia vermicola glutamine synthetase or a Photorhabdus luminescens glutamine synthetase.
Item 4 specifies for the mammalian expression vector of any one of items 1 to 3 that the bacterial glutamine synthetase comprises an amino acid sequence having at least 85% sequence identity with the amino acid sequence of SEQ ID NO: 1, and a mutation selected from the group consisting of E130X, F226Y, R345A and combinations thereof, wherein X is any amino acid, preferably wherein the mutation in amino acid position E130 is a substitution with an aromatic or hydrophobic amino acid, more preferably selected from the group consisting of Y, W, F, A, G, V, L, M and I.
Item 5 specifies for the mammalian expression vector of any one of items 1 to 4 that the bacterial glutamine synthetase comprises an amino acid sequence having at least 95% sequence identity with the amino acid sequence of SEQ ID NO: 1 or SEQ ID NO: 14, and a mutation selected from the group consisting of E130X, F226Y, R345A and combinations thereof, wherein X is any amino acid, preferably wherein the mutation in amino acid position E130 is a substitution with an aromatic or hydrophobic amino acid, more preferably selected from the group consisting of Y, W, F, A, G, V, L, M and I.
Item 6 further specifies the mammalian expression vector of item 4 or 5 in that the mutation is E130X and/or F226Y and/or R345A, and wherein E130X is E130F or E130G.
Item 7 further specifies the mammalian expression vector of any one of items 1 to 6 in that the polynucleotide encoding the bacterial glutamine synthetase is operably linked to a mammalian promoter.
Item 8 further specifies the mammalian expression vector of any one of items 1 to 7 in that the mammalian expression vector further comprises at least one polynucleotide encoding a protein of interest and/or a non-coding RNA, preferably an expression cassette comprising at least one polynucleotide encoding a protein of interest and/or a non-coding RNA.
Item 9 further specifies the mammalian expression vector according to any one of items 1 to 8 in that the protein of interest is a therapeutic protein, preferably selected from the group consisting of a cytokine, a hormone, a fusion protein, an antibody, an antibody-derived molecule and an antibody mimetic.
Item 10 further specifies the mammalian expression vector according to item 9 in that the protein of interest is an antibody, preferably wherein expression vector comprises a polynucleotide comprising a coding sequence for a variable region of the heavy chain and/or a coding sequence for a variable region of the light chain of the antibody.
Item 11 further specifies the mammalian expression vector according to any one of items 8 to 10 in that the mammalian expression vector comprises a multicistronic expression cassette and/or multiple expression cassettes, preferably wherein the expression vector comprises multiple expression cassettes.
Item 12 further specifies the mammalian expression vector of any one of items 8 to 11 in that the mammalian expression vector is for stable transfection and the integrating part of the vector comprises the polynucleotide encoding the bacterial glutamine synthetase and at least one polynucleotide encoding the protein of interest and/or the non-coding RNA.
Item 13 further specifies the mammalian expression vector according to any one of items 1 to 12 in that the mammalian expression vector is a plasmid, a Bacterial Artificial Chromosome (BAC) or a viral vector.
Item 14 provides a nucleic acid sequence comprising a polynucleotide encoding a bacterial glutamine synthetase operably linked to a mammalian promoter, optionally further comprising at least one polynucleotide encoding a protein of interest and/or a non-coding RNA.
Item 15 further specifies the nucleic acid sequence of item 14 in that the bacterial glutamine synthetase comprises an amino acid sequence having at least 85% sequence identity with the amino acid sequence of SEQ ID NO: 1, and a mutation selected from the group consisting of E130X, F226Y, R345A and combinations thereof, wherein X is any amino acid, preferably wherein the mutation in amino acid position E130 is a substitution with an aromatic or hydrophobic amino acid, more preferably selected from the group consisting of Y, W, F, A, G, V, L, M and I.
Item 16 further specifies the nucleic acid sequence of item 15 in that the bacterial glutamine synthetase comprises an amino acid sequence having at least 95% sequence identity with the amino acid sequence of SEQ ID NO: 1, and a mutation selected from the group consisting of E130X, F226Y, R345A and combinations thereof, wherein X is any amino acid, preferably wherein the mutation in amino acid position E130 is a substitution with an aromatic or hydrophobic amino acid, more preferably selected from the group consisting of Y, W, F, A, G, V, L, M and I.
Item 17 further specifies the nucleic acid sequence of item 15 in that the bacterial glutamine synthetase comprises an amino acid sequence having at least 95% sequence identity with the amino acid sequence of SEQ ID NO: 14, and a mutation selected from the group consisting of E130X, F226Y, R345A and combinations thereof, wherein X is any amino acid, preferably wherein the mutation in amino acid position E130 is a substitution with an aromatic or hydrophobic amino acid, more preferably selected from the group consisting of Y, W, F, A, G, V, L, M and I.
Item 18 further specifies the nucleic acid sequence of any one of items 15 to 17 in that the mutation is E130X and/or F226 and/or R345A and wherein E130X is E130F or E130G
Item 19 provides a bacterial glutamine synthetase comprising an amino acid sequence having at least 85% sequence identity with the amino acid sequence of SEQ ID NO: 1 and a mutation selected from the group consisting of E130X, F226Y, R345A and combinations thereof, wherein X is any amino acid, preferably wherein the mutation in amino acid position E130 is a substitution with an aromatic or hydrophobic amino acid, more preferably selected from the group consisting of Y, W, F, A, G, V, L, M and I.
Item 20 provides a bacterial glutamine synthetase from Providencia vermicola comprising an amino acid sequence having at least 95% sequence identity with the amino acid sequence of SEQ ID NO: 1 and a mutation selected from the group consisting of E130X, F226Y, R345A and combinations thereof, wherein X is any amino acid, preferably wherein the mutation in amino acid position E130 is a substitution with an aromatic or hydrophobic amino acid, more preferably selected from the group consisting of Y, W, F, A, G, V, L, M and I.
Item 21 provides a bacterial glutamine synthetase from Photorhabdus luminescens comprising an amino acid sequence having at least 95% sequence identity with the amino acid sequence of SEQ ID NO: 14 and a mutation selected from the group consisting of E130X, F226Y, R345A and combinations thereof, wherein X is any amino acid, preferably wherein the mutation in amino acid position E130 is a substitution with an aromatic or hydrophobic amino acid, more preferably selected from the group consisting of Y, W, F, A, G, V, L, M and I.
Item 22 provides a mammalian host cell comprising the expression vector of any one of items 1 to 13, the nucleic acid sequence of any one of items 14 to 18, or a nucleic acid sequence encoding the bacterial glutamine synthetase of any one of items 19 to 21, preferably wherein the mammalian host cell is a rodent cell, more preferably a CHO cell and/or (b) a GS gene knockout cell.
Item 23 further specifies the mammalian host cell of item 22 in that it comprises a polynucleotide encoding a bacterial glutamine synthetase and at least one polynucleotide encoding a protein of interest and/or the non-coding RNA co-integrated into the host cell genome.
Item 24 provides a method for preparing a cell stably expressing a protein of interest and/or a non-coding RNA, comprising (a) introducing the expression vector according to items 1 to 13 or the nucleic acid of any one of items 14 to 18 comprising a polynucleotide encoding a bacterial glutamine synthetase and at least one polynucleotide encoding a protein of interest and/or a non-coding RNA into a mammalian host cell, such as a CHO cell; and (b) culturing the mammalian host cell in a medium comprising no glutamine under conditions to select for the bacterial glutamine synthetase, wherein the at least one polynucleotide encoding a protein of interest and/or the non-coding RNA is co-integrated with the polynucleotide encoding the bacterial glutamine synthetase into the host cell genome.
Item 25 further specifies the method of item 24, to comprise (c) a step of isolating a single clone for clonal expansion to prepare a monoclonal cell line.
Item 26 provides a method of producing a protein of interest, comprising (a) introducing the mammalian expression vector according to items 1 to 13 or the nucleic acid of any one of items 14 to 18 into a mammalian host cell, preferably into a mammalian host cell, such as a CHO cell, wherein the expression vector comprises a polynucleotide encoding a bacterial glutamine synthetase operably linked to a mammalian promoter and at least one polynucleotide encoding a protein of interest; (b) culturing the mammalian host cell in a medium comprising no glutamine under conditions to select for the bacterial glutamine synthetase, wherein the at least one polynucleotide encoding a protein of interest is co-integrated with the polynucleotide encoding the bacterial glutamine synthetase into the host cell genome; (c) optionally isolating a single clone for clonal expansion to prepare a monoclonal cell line; (d) culturing the mammalian host cell under conditions to produce the protein of interest; and (e) harvesting and optionally purifying the protein of interest.
Item 27 further specifies the method of any one of items 24 to 26 in that the mammalian expression vector or the nucleic acid is introduced by transfection or transduction.
Item 28 further specifies the method of item 27 in that the mammalian expression vector or the nucleic acid is introduced by stable transfection.
Item 29 provides a method of producing a protein of interest, comprising (a) providing the mammalian host cell of item 22 or 23, comprising a polynucleotide encoding a bacterial glutamine synthetase operably linked to a mammalian promoter and at least one polynucleotide encoding a protein of interest; (b) culturing the mammalian host cell under conditions to produce the protein of interest; and (c) harvesting and optionally purifying the protein of interest.
Item 30 further specifies the method of any one of items 24 to 29, wherein (a) the mammalian host cell is a GS gene knockout cell; and/or (b) culturing the mammalian host cell in a medium comprising no glutamine under conditions to select for the bacterial glutamine synthetase in step (b) comprises the addition of the GS inhibitor methionine sulfoximine (MSX).
Item 31 further specifies the method of any one of items 24 to 30 in that the mammalian host cell is a rodent cell, preferably a CHO cell.
Item 32 further specifies the method of any one of items 24 to 30 in that the cell or cell line is generated with increased selection stringency and/or has increased genetic stability and/or has higher productivity compared to a cell or cell line generated with a glutamine synthetase from Cricetulus griseus having the amino acid sequence of SEQ ID NO: 2.
Item 33 provides a kit comprising the expression vector of any one of items 1 to 13 and a cell culture medium not comprising glutamine.
Item 34 provides a use of a bacterial glutamine synthetase as a selection marker in a mammalian cell, preferably wherein the bacterial glutamine synthetase comprises an amino acid sequence having at least 85% sequence identity with the amino acid sequence of SEQ ID NO: 1.
Item 35 further specifies the use of item 34 in that the bacterial glutamine synthetase is derived from bacteria of the order Enterobacterales and of the family Monganellaceae, more preferably of a Providencia species or a Photorhabdus species, even more preferably of Providencia vermicola or Photorhabdus luminescens.
Item 36 further specifies the use of item 35 in that the bacterial glutamine synthetase is (a) derived from a Providencia species and/or comprises an amino acid sequence having at least 95% sequence identity with the amino acid sequence of SEQ ID NO: 1; more preferably wherein the bacterial glutamine synthetase is a Providencia vermicola glutamine synthetase.
Item 37 further specifies the use of any one of items 34 to 36 in that the bacterial glutamine synthetase comprises an amino acid sequence having at least 95% sequence identity with the amino acid sequence of SEQ ID NO: 1, and a mutation selected from the group consisting of E130X, F226Y, R345A and combinations thereof, wherein X is any amino acid, preferably wherein the mutation in amino acid position E130 is a substitution with an aromatic or hydrophobic amino acid, more preferably selected from the group consisting of Y, W, F, A, G, V, L, M and I.
Item 38 further specifies the use of item 34 in that the bacterial glutamine synthetase is derived from a Photorhabdus species and/or comprises an amino acid sequence having at least 95% sequence identity with the amino acid sequence of SEQ ID NO: 1; more preferably wherein the bacterial glutamine synthetase is a Photorhabdus luminescens glutamine synthetase.
Item 39 further specifies the use of any one of items 34, 35 and 38 in that the bacterial glutamine synthetase comprises an amino acid sequence having at least 95% sequence identity with the amino acid sequence of SEQ ID NO: 14, and a mutation selected from the group consisting of E130X, F226Y, R345A and combinations thereof, wherein X is any amino acid, preferably wherein the mutation in amino acid position E130 is a substitution with an aromatic or hydrophobic amino acid, more preferably selected from the group consisting of Y, W, F, A, G, V, L, M and I.
Item 40 further specifies the use of item 37 or 39, wherein the mutation is E130X and/or F226Y and/or R345A, and wherein E130X is E130F or E130G.

### Examples

### Linearization of Plasmids for Transfection

The plasmids used for stable transfection comprise an CMV driven antibody transcription cassette, an ampicillin transgene and a glutamine synthetase transgene as metabolic selection marker. The plasmids were linearized for transfection using PvuI (single cut in the ampicillin transgene). The restriction digest of 20 μg plasmid DNA was conducted at 37° C. for 3 h with PvuI (NEB) according to the manufacturers protocol. The linearized plasmid DNA was purified (Qiagen Plasmid Maxi Kit) according to the manufacturers protocol. The final DNA concentration was determined via Nanodrop spectral photometer.

### Host Cell Cultivation

CHO-K1-GS knock out cell line host cell (also referred to as CHO-K1-GS KO or simply CHO-K1-GS), harboring a genomic knockout of the endogenous gluthamine synthetase gene, was cultivated in host cell medium with added L-glutamine. The cultivation of the host cell was started with a seeding density of 3×10E05 cells per mL. The growth conditions were set to 36.5° C. and 5% CO₂ in a shaking incubator with 120 rpm in shake flasks. Determination of cell density and viability took place in the Cedex HiRes® cell count analyzer.

### Transfection of Linearized Plasmids in CHO

One day before transfection, the host cells were seeded with a cell density of 0.8×10E06 cells/mL in shake Flask. On the day of transfection cell density and viability were determined and the required amount of cells for transfection was centrifuged for 7 minutes with 750×G. The supernatant was discarded. 10 μg of linearized plasmid DNA per transfection were transfected using the Neon® transfection system (Invitrogen) and electoporation. The electroporation cuvette was filled with 3 mL of electroporation buffer E2, resuspended cells (5×10E6) in 89 μl of buffer R and mixed with 10 μg of linearized plasmid. The transfection was performed using 1500 Volt, 10 mS and a pulse of 2. Transfected cells were transferred in 5 ml prewarmed host cell medium in T25 ml flasks and incubated with 8% CO₂ and 37° C. for at least 24 h.

### Selection of Stable CHO Pools

24 h after transfection, cells were transferred into selection medium (medium without L-glutamine). 10 mL of selection medium was prewarmed for every pool in T75 Flasks. Two stable pools were cultivated for each transfection. Cells were centrifuged (for 7 minutes with 750×G) and resuspended in 20 mL of selection medium and incubated with 8% CO₂ at 37° C. During selection, cells were monitored by microscopy. Additional selection medium (5 mL) was added after 7 days.

### Passaging and Production Run of Stable Pools

Once cells reached viability of at least 70% and a doubling time of 48 hours or less, selection phase was considered successful. After selection, cells were passaged every two to three days starting with 3×10E05 cells per mL in 30 mL total volume of medium (not containing L-glutamine) in 125 mL shake flasks at 36.5° C. and 5% CO₂ at 120 rpm shaking. Samples for titer measurements were taken regularly. If antibody titer was stable for at least 14 days, indicating phenotypic stability, an at least 7 days production run was started.

For the production run cells were seeded with a density of 7×10E05 viable cells per mL in 30 mL total volume basal medium without glutamine in shake flasks and cells were cultivated at 34.5° C., 5% CO₂ and 120 rpm shaking. During the at least 7 day production run, cells were counted and samples for pH, glucose and titer measurement were collected daily to adjust pH and glucose feeding if necessary. Starting from day 2 a daily feed (w/o glutamine) was supplemented. The pH was determined using the RAPIDlab 348Ex® and glucose levels were measured via an EKF diagnostics device. During and/or at the end of culture, cell culture medium supernatant was analyzed following centrifugation for antibody titers using a ForteBio Octet® device with protein A Biosensors. Dilution of samples and standard curve were processed in the same production medium.

### Example 1: Antibody Production in CHO Cells Using CHO Wildtype Glutamine Synthetase as Metabolic Selection Marker

CHO-K1-GS cells once transfected with a vector carrying a glutamine synthetase from Cricetulus griseus (CHO wildtype glutamine synthetase (GS), SEQ ID NO: 2) selection marker can survive selection under cultivation in medium without supplementation of L-glutamine, when the transgene vector is stably integrated into the genome of the cell.

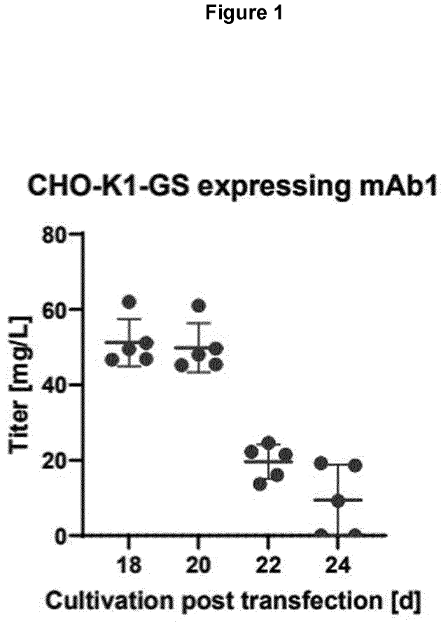

CHO-K1-GS cells were transfected with a vector carrying the expression cassettes of a monoclonal antibody 1 (mAb1) and a CHO wildtype GS. Following selection, stable CHO pools were passaged as described above and samples for titer measurements of mAb1 were taken regularly. Under the applied experimental conditions, the stable CHO pools (transfected with wildtype CHO GS) remained stable and productive for ~20 days post transfection with decreasing levels of productivity (titer) thereafter (FIG. 1).

### Example 2: Antibody Production in CHO Cells Using Providencia vermicola GS and Other Bacterial GS as Selection Marker

To functionally test novel bacterial glutamine synthetase variants, selection stringency (duration to reach >70% viability after transfection), amount of productive passages and specific productivity were measured over time. For CHO pools, which remain productive after prolonged passaging, a 7-day production run in shaking flasks was subsequently conducted in a controlled process set-up to additionally evaluate cell culture process parameters.

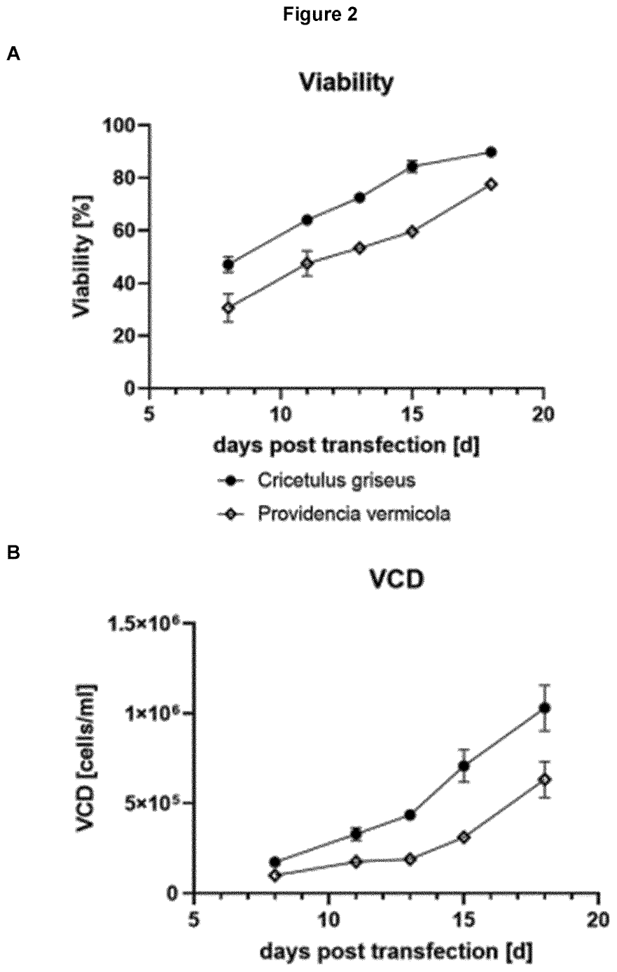

CHO-K1-GS cells were transfected with a vector carrying the expression cassette of mAb1 and the glutamine synthetase from Cricetulus griseus (SEQ ID NO: 2) or Providencia vermicola (SEQ ID NO: 1) and viability, viable cell count (VCD) and productivity were measured over time during the selection phase. Surprisingly, CHO cells transfected with the bacterial Class-I GS from Providencia vermicola survived the selection process, suggesting that this prokaryotic metabolic selection marker is functional in CHO cells. Cells transfected with the Providencia GS took longer to fully recover from selection (FIG. 2A) and cell growth was slower (FIG. 2B) in comparison to the CHO GS, suggesting that the selection stringency is increased.

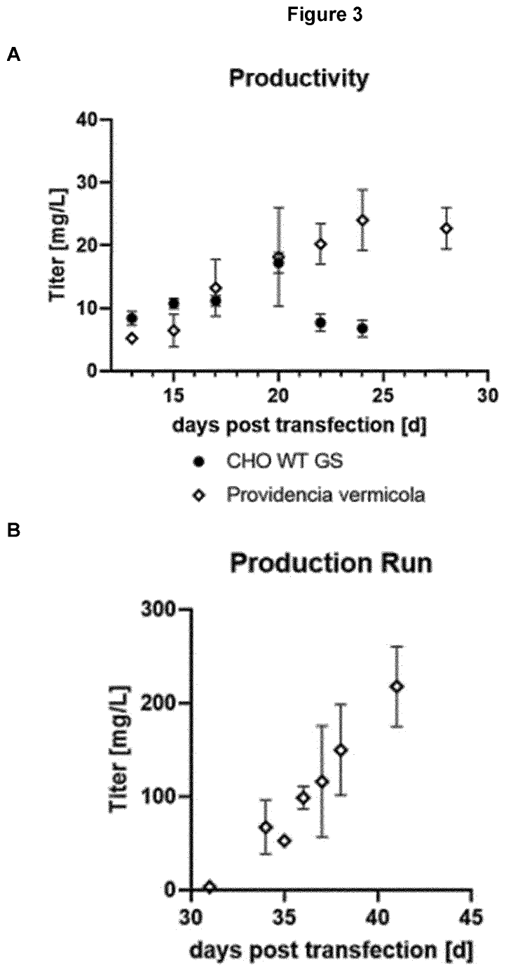

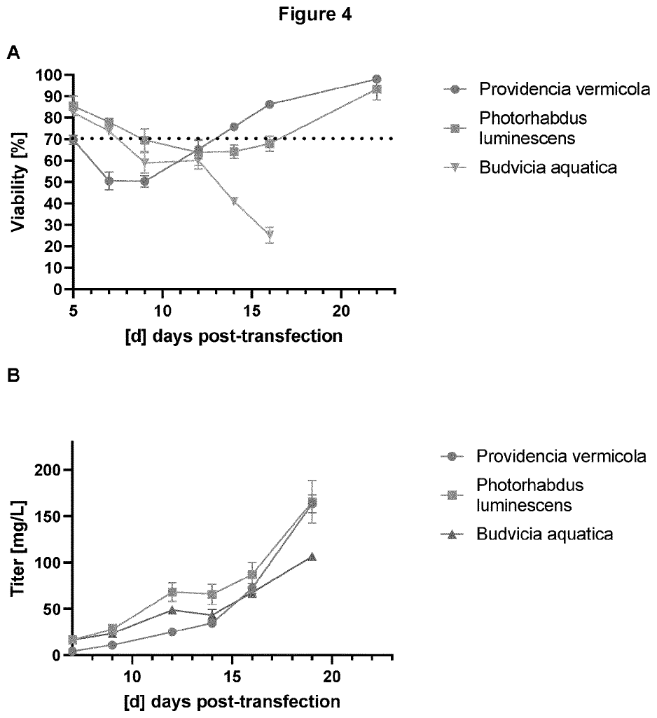

The titer was measured during cell selection, indicating that the productivity of cells harboring the CHO GS gene rapidly declined. Cells transfected with the Providencia GS were stably expressing mAb1 even after the selection process (FIG. 3A). An additional at least 7-day production run was conducted after selection and passaging, suggesting that the phenotypic stability of the cells transfected with the Providencia GS is significantly higher compared to CHO GS (FIG. 3B). Cells transfected with the CHO GS were not transferred into a production run due to lost productivity.

Overall, CHO cells transfected with the Providencia vermicola glutamine synthetase showed an increased selection stringency and significantly higher productivity. Moreover, cell pools show an increased phenotypic stability and remain productive for at least 40 days, whereas cells transfected with CHO GS completely lost their productivity after ~20 days post transfection.

In addition to GS from Providencia vermicola, a total of 10 prokaryotic GS have been tested of which 9 were derived from bacteria and one derived from archea (Methanocaldococcus janaschii). Six were found to be functional and 4 were found to be non-functional in CHO cells. Functional was defined as survived the selection process and gave yield to antibody producing cells. The results are summarized below in Table B with (+++) indicting excellence performance superior to WT CHO GS, (++) indicating moderate performance similar to WT CHO GS and (+) indicating modest performance, clearly inferior to WT CHO GS. The majority of bacterial GS that were at least successfully used for selection belong to the order Enterobacterales and the family Morganellaceae and/or have at least 80% sequence identity with GS from Providencia vermicola. However, most of these bacterial GS turned out to show moderate performance, clearly inferior to WT CHO GS when considering selection stringency and genetic stability, except for GS from Photorhabdus luminescence, which indicated excellence performance (selection stringency/genetic stability) superior to WT CHO GS.

TABLE B

| | | | Selection | Homology |
|---|---|---|---|---|
| | | Functional | stringency/ | to |
| Glutamine | | in CHO | genetic | Providencia |
| synthethase | Classification | cells | stability | sp GS [%] |
| Providencia | Enterobacterales; | Yes | +++/+++ | 100 |
| vermicola | Morganellaceae; | | | |
| | Providencia | | | |
| Bacillus Subtilis | Bacillales; Bacillaceae; | No | | 40.5 |
| (SEQ ID NO: 10) | Bacillus; Bacillus subtilis | | | |
| | group | | | |
| Methanocaldococcus | Methanococcales; | No | | 38.3 |
| janaschii | Methanocaldococcaceae; | | | |
| (SEQ ID NO: 11) | Methanocaldococcus | | | |
| Corynebacterium | Mycobacteriales; | Yes | ++/+ | 51 |
| glutamicum | Corynebacteriaceae; | | (selection | |
| (SEQ ID NO: 12) | Corynebacterium | | not robust) | |
| Cryobacterium soli | Microbacteriaceae; | Yes | +/+ | 54 |
| (SEQ ID NO: 13) | Cryobacterium | | (selection not | |
| | | | robust and | |
| | | | genetically | |
| | | | instable) | |
| Photorhabdus | Enterobacterales; | Yes | +++/+++ | 87.2 |
| luminescens | Morganellaceae; | | | |
| (SEQ ID NO: 14) | Photorhabdus | | | |
| Moellerella | Enterobacterales; | Yes | +/+ | 92.1 |
| wisconsensis | Morganellaceae; | | (selection not | |
| (SEQ ID NO: 15) | Moellerella | | robust and | |
| | | | genetically | |
| | | | instable) | |
| Edwardsiella | Enterobacterales; | No | | 85.3 |
| (SEQ ID NO: 16) | Hafniaceae | | | |
| Budvicia aquatica | Enterobacterales; | Yes | ++/+ | 87.8 |
| (SEQ ID NO: 17) | Budviciaceae; Budvicia | | (genetically | |
| | | | instable) | |
| Brenneria roseae | Enterobacterales; | No | | 84.9 |
| (SEQ ID NO: 18) | Pectobacteriaceae; | | | |
| | Brenneria | | | |
| Arsenophonus sp. | Enterobacterales; | Yes | ++/+ | 83.8 |
| (SEQ ID NO: 19) | Morganellaceae; | | (genetically | |
| | Arsenophonus; | | instable) | |
| | unclassified | | | |
| | Arsenophonus | | | |

Selection stringency is analysed by measuring recovery of viability of >70% and genetic stability is demonstrated by stable productivity over a certain period of time, such as more than 20 days post-transfection. Representative date for selections experiments monitoring viability of stable CHO pools using GS from Providencia vermicola, Photorhabdus luminescens and Budvicia aquatica as selection marker are shown in FIG. 4A and for productivity of CHO pools transfected with these bacterial GS are shown in FIG. 4B. While GS from Budvicia aquatica was functional in CHO cells, selection was not robust. In contrast, GS from Photorhabdus luminescens remained functionally active and showed even stronger attenuated activity compared GS from Providencia vermicola (FIG. 4A). Stable pools transfected with GS from Photorhabdus luminescens recovered 16 to 17 days post-transfection (reaching a viability of >70%), compared to 13 days post-transfection for GS from Providencia vermicola. Productivity was comparable for GS from Photorhabdus luminescens and Providencia vermicola, although slightly delayed for GS from Photorhabdus luminescens, and strongly reduced for GS from Budvicia aquatica (FIG. 4B).

These experiments demonstrate for the first time that bacterial GS, such as from Providencia vermicola, can be used as selection marker in mammalian cells and further that other bacterial GS closely related bacterial GS from the order Enterobacterales and the family Morganellaceae and/or having at least 85% sequence identity with the amino acid sequence of GS from Providencia vermicola (SEQ ID NO: 1) can be identified, such as GS from Photorhabdus luminescence.

### Example 3: Antibody Production in CHO Cells Using Providencia vermicola Wildtype GS and E130F or E130G Mutants as Selection Marker

Furthermore, two distinct point mutations of Providencia vermicola GS (E130F and E130G) were tested in selection and productions experiments. CHO-K1-GS cells were transfected with a vector carrying expression cassettes for mAb1 and wildtype glutamine synthetase from Providencia vermicola (SEQ ID NO: 1) or glutamine synthetase from Providencia vermicola comprising mutation E130F (SEQ ID NO: 4) or E130G (SEQ ID NO: 5).

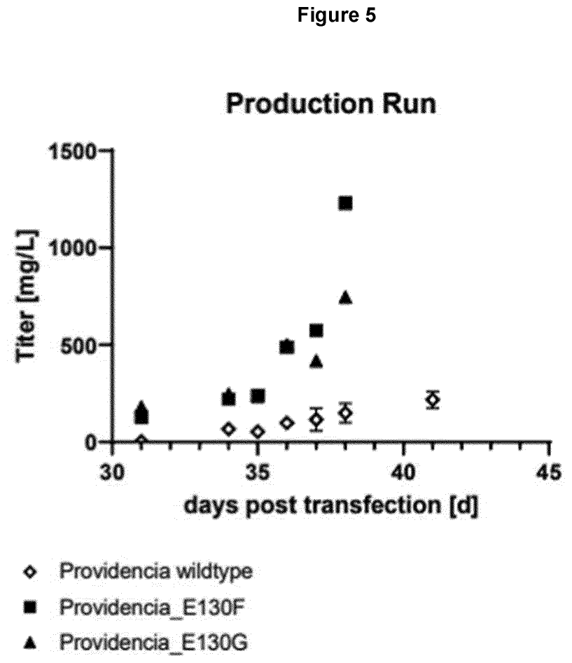

Both mutations seem to further extend the degree of attenuation, leading to prolonged durations for the cells to grow out of selection compared to wildtype Providencia vermicola glutamine synthetase and both demonstrated phenotypic stability. Subsequent production runs resulted in significantly improved overall titer (FIG. 5).

### Example 4: Antibody Production and VCD in CHO Cells Using Providencia vermicola Wildtype GS and F226Y Mutant as Selection Marker

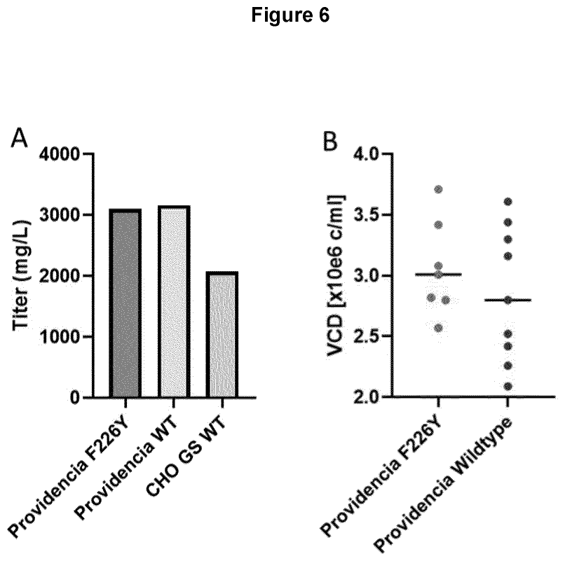

CHO-K1-GS cells were transfected with a vector carrying the expression cassettes for mAb1 and CHO wildtype GS (CHO WT GS), Providencia wildtype GS (SEQ ID NO: 1) or Providencia GS F226Y mutant (SEQ ID NO: 6). After stable pool generation and single cell deposition, top clones were subjected to shaking flask experiment head-to-head with CHO WT GS. Both Providencia wildtype GS and Providencia GS F226 outperformed CHO wildtype GS, showing about 50% higher titers (mg/L) compared to CHO wildtype GS clones (FIG. 6A). Compared Providencia wildtype GS, cell clones with Providencia GS F226Y showed further increased growth behavior with higher viable cell densities (FIG. 6B). This may provide an advantage for biopharmaceutical production and particularly for combinations with E130 mutations.

### Example 5: Antibody Production and Viability in CHO Cells Using Providencia vermicola Wildtype GS and R345A or R360A Mutants as Selection Marker

Furthermore, two additional point mutants of the Providencia vermicola glutamine synthetase were tested and assessed in the generation of stable antibody producing CHO expression pools. Wild type glutamine synthetase was tested head-to-head against variants with mutations in the catalytic domain of the selection marker.

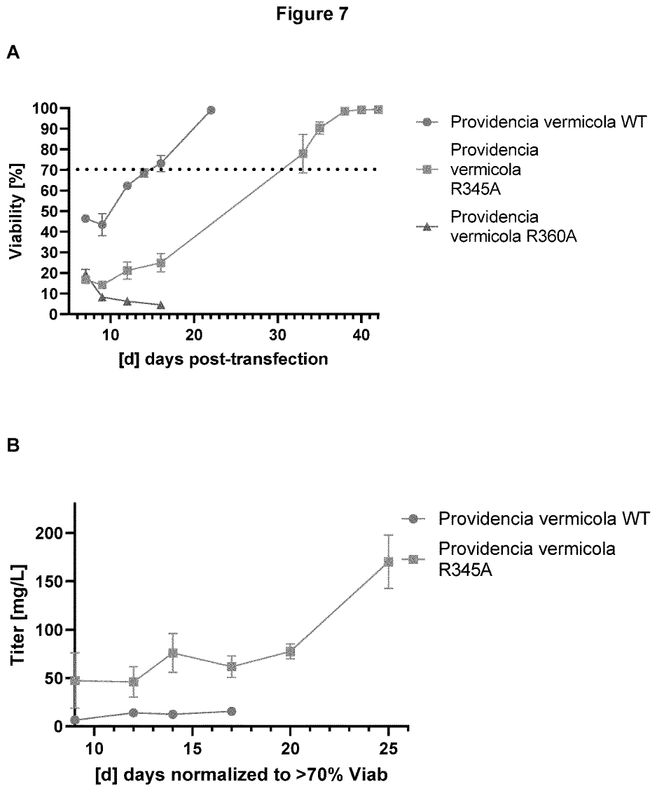

While R360A lost functionality and was unable to recover pools from the selection experiments, R345A remained functionally active but with strongly attenuated activity (FIG. 7A). Stable pools transfected with R345A recovered 31 days post-transfection (reaching a viability of >70%) in comparison to the wildtype protein, which recovered in ~15 days. Interestingly, the strongly attenuated variant R345A (SEQ ID NO: 20) resulted in a 5-10 fold increase in productivity (FIG. 7B).

Overall, the results from Examples 3, 4 and 5 demonstrate that bacterial GS, such as from Providencia vermicola, can be further attenuated and thereby improved as selection marker by introducing one or more point-mutations at a conserved residue involved in substrate binding, such as E130F, E130G, F226Y and/or R345A. Since these residues E130, F226 and R345 are also present in GS from Photorhabdus luminescence (amino acid sequence of SEQ ID NO: 14), it is expected that these mutations similarly further attenuate this bacterial GS and thereby improve this GS as selection marker. Particularly the mutation F226Y showed further increased growth behavior with higher viable cell densities (FIG. 6B). This may provide an advantage for biopharmaceutical production and particularly for combinations with E130 mutations or with the R345A mutation.

### SEQUENCE LISTING

| SEQ ID NO: | description |
|---|---|
| 1 | Wild type Providencia vermicola glutamine synthetase |
| 2 | Wild type Cricetulus griseus glutamine synthetase |
| 3 | Providencia vermicola glutamine synthetase E130X |
| 4 | Providencia vermicola glutamine synthetase E130F |
| 5 | Providencia vermicola glutamine synthetase E130G |
| 6 | Providencia vermicola glutamine synthetase F226Y |
| 7 | Providencia vermicola glutamine synthetase E130X and F226Y |
| 8 | Providencia vermicola glutamine synthetase E130F and F226Y |
| 9 | Providencia vermicola glutamine synthetase E130G and F226Y |
| 10 | Wild type Bacillus Subtilis glutamine synthetase |
| 11 | Wild type Methanocaldococcus janaschii glutamine synthetase |
| 12 | Wild type Corynebacterium glutamicum glutamine synthetase |
| 13 | Wild type Cryobacterium soli glutamine synthetase |
| 14 | Wild type Photorhabdus luminescens glutamine synthetase |
| 15 | Wild type Moellerella wisconsensis glutamine synthetase |
| 16 | Wild type Edwardsiella glutamine synthetase |
| 17 | Wild type Budvicia aquatica glutamine synthetase |
| 18 | Wild type Brenneria roseae glutamine synthetase |
| 19 | Wild type Arsenophonus sp. glutamine synthetase |
| 20 | Providencia vermicola glutamine synthetase R345A |
| 21 | Providencia vermicola glutamine synthetase F226Y and R345A |
| 22 | Providencia vermicola glutamine synthetase E130X and R345A |
| 23 | Photorhabdus luminescense glutamine synthetase E130X |
| 24 | Photorhabdus luminescens glutamine synthetase F226Y |
| 25 | Photorhabdus luminescens glutamine synthetase R345A |
| 26 | Photorhabdus luminescens glutamine synthetase E130X and F226Y |
| 27 | Photorhabdus luminescens glutamine synthetase E130X and R345A |
| 28 | Photorhabdus luminescens glutamine synthetase F226Y and R345A |
| 29 | Providencia vermicola glutamine synthetase E130X and F226Y and R345A |
| 30 | Photorhabdus luminescens glutamine synthetase E130X and F226Y and |
| | R345A |

1. A mammalian expression vector comprising a polynucleotide encoding a bacterial glutamine synthetase as a selection marker, wherein the bacterial glutamine synthetase comprises an amino acid sequence having at least 85% sequence identity with the amino acid sequence of SEQ ID NO: 1.

2. The mammalian expression vector of claim 1, wherein the bacterial glutamine synthetase is derived from bacteria of the order Enterobacterales and of the family of Morganellaceae.

3. The mammalian expression vector of claim 1, wherein the bacterial glutamine synthetase comprises an amino acid sequence having at least 85% sequence identity with the amino acid sequence of SEQ ID NO: 1, and a mutation selected from the group consisting of E130X, F226Y, R345A and combinations thereof, wherein X is any amino acid.

4. The mammalian expression vector of claim 1, wherein the bacterial glutamine synthetase comprises an amino acid sequence having at least 95% sequence identity with the amino acid sequence of SEQ ID NO: 1 or SEQ ID NO: 14, and a mutation selected from the group consisting of E130X, F226Y, R345A and combinations thereof, wherein X is any amino acid.

5. The mammalian expression vector of claim 1, wherein the mammalian expression vector further comprises at least one polynucleotide encoding a protein of interest and/or a non-coding RNA.

6. The mammalian expression vector according to claim 5, wherein the protein of interest is a therapeutic protein.

7. A nucleic acid sequence comprising a polynucleotide encoding a bacterial glutamine synthetase comprising an amino acid sequence having at least 85% sequence identity with the amino acid sequence of SEQ ID NO: 1 operably linked to a mammalian promoter, optionally further comprising at least one polynucleotide encoding a protein of interest and/or a non-coding RNA.

8. The nucleic acid sequence of claim 7, wherein the bacterial glutamine synthetase comprises an amino acid sequence having at least 85% sequence identity with the amino acid sequence of SEQ ID NO: 1, and a mutation selected from the group consisting of E130X, F226Y, R345A and combinations thereof, wherein X is any amino acid.

9. A bacterial glutamine synthetase comprising an amino acid sequence having at least 85% sequence identity with the amino acid sequence of SEQ ID NO: 1 and a mutation selected from the group consisting of E130X, F226Y, R345A and combinations thereof, wherein X is any amino acid.

10. A mammalian host cell comprising the expression vector of claim 1.

11. A method for preparing a cell stably expressing a protein of interest and/or a non-coding RNA, comprising
(a) introducing the expression vector according to claim 1 comprising a polynucleotide encoding a bacterial glutamine synthetase and at least one polynucleotide encoding a protein of interest and/or a non-coding RNA into a mammalian host cell; and
(b) culturing the mammalian host cell in a medium comprising no glutamine under conditions to select for the bacterial glutamine synthetase, wherein the at least one polynucleotide encoding a protein of interest and/or a non-coding RNA is co-integrated with the polynucleotide encoding the bacterial glutamine synthetase into the host cell genome.

12. The method of claim 11, further comprising
(c) a step of isolating a single clone for clonal expansion to prepare a monoclonal cell line.

13. A method of producing a protein of interest, comprising
(a) introducing the mammalian expression vector according to claim 1 into a mammalian host cell, wherein the expression vector comprises a polynucleotide encoding a bacterial glutamine synthetase operably linked to a mammalian promoter and at least one polynucleotide encoding a protein of interest;
(b) culturing the mammalian host cell in a medium comprising no glutamine under conditions to select for the bacterial glutamine synthetase, wherein the at least one polynucleotide encoding a protein of interest is co-integrated with the polynucleotide encoding the bacterial glutamine synthetase into the host cell genome;
(c) optionally isolating a single clone for clonal expansion to prepare a monoclonal cell line;
(d) culturing the mammalian host cell under conditions to produce the protein of interest; and
(e) harvesting and optionally purifying the protein of interest.

14. A method of producing a protein of interest, comprising
(a) providing the mammalian host cell of claim 10, comprising a polynucleotide encoding a bacterial glutamine synthetase operably linked to a mammalian promoter and at least one polynucleotide encoding a protein of interest;
(b) culturing the mammalian host cell under conditions to produce the protein of interest; and
(c) harvesting and optionally purifying the protein of interest.

15. (canceled)

16. A kit comprising the expression vector of claim 1 and a cell culture medium not comprising glutamine.

17. (canceled)

18. A mammalian host cell comprising the nucleic acid sequence of claim 7.

19. A mammalian host cell comprising a nucleic acid sequence encoding the bacterial glutamine synthetase of claim 9.

20. A method for preparing a cell stably expressing a protein of interest and/or a non-coding RNA, comprising
(a) introducing the nucleic acid of claim 7 comprising the polynucleotide encoding a bacterial glutamine synthetase and at least one polynucleotide encoding a protein of interest and/or a non-coding RNA into a mammalian host cell; and
(b) culturing the mammalian host cell in a medium comprising no glutamine under conditions to select for the bacterial glutamine synthetase, wherein the at least one polynucleotide encoding a protein of interest and/or a non-coding RNA is co-integrated with the polynucleotide encoding the bacterial glutamine synthetase into the host cell genome.

21. A method of producing a protein of interest, comprising
(a) introducing the nucleic acid of claim 7 into a mammalian host cell, wherein the nucleic acid comprises the polynucleotide encoding a bacterial glutamine synthetase operably linked to a mammalian promoter and at least one polynucleotide encoding a protein of interest;
(b) culturing the mammalian host cell in a medium comprising no glutamine under conditions to select for the bacterial glutamine synthetase, wherein the at least one polynucleotide encoding a protein of interest is co-integrated with the polynucleotide encoding the bacterial glutamine synthetase into the host cell genome;
(c) optionally isolating a single clone for clonal expansion to prepare a monoclonal cell line;
(d) culturing the mammalian host cell under conditions to produce the protein of interest; and
(e) harvesting and optionally purifying the protein of interest.

22. A method of producing a protein of interest, comprising
(a) providing the mammalian host cell of claim 18, comprising a polynucleotide encoding a bacterial glutamine synthetase operably linked to a mammalian promoter and at least one polynucleotide encoding a protein of interest;
(b) culturing the mammalian host cell under conditions to produce the protein of interest; and
(c) harvesting and optionally purifying the protein of interest.

23. A method of producing a protein of interest, comprising
(a) providing the mammalian host cell of claim 19, comprising a polynucleotide encoding a bacterial glutamine synthetase operably linked to a mammalian promoter and at least one polynucleotide encoding a protein of interest;
(b) culturing the mammalian host cell under conditions to produce the protein of interest; and
(c) harvesting and optionally purifying the protein of interest.


SEQUENCE LISTING (30 sequences)

SEQ ID NO: 1 - protein, 469 aa, Providencia vermicola
  REGION 1..469: note = Wild type Providencia vermicola glutamine synthetase
MSAEHILSLIEEHNVRFIDLRFTDTKGKEQHITIPAHQVDEDFFEEGQMFDGSSFGGWKGINESDMVLMPDPSTAMIDPFFADITLIIRCDILEPGTMQGYGRDPRSISKRAEDFLRSSGIADVVLFGPEPEFFVFDDIRFGNSMHSSYYHIDDIEGAWNTGTKYEGGNKGHRPSVKGGYFPVPPVDSSQDLRSAMCTTMEDLGLVVEAHHHEVATAGQNEIATRFNTMTKKADETQVYKYVVHNVAHAFGKTATFMPKPLVGDNGSGMHCHMSLSKDGVNLFAGDKYGGLSEMALYYIGGIIKHARALNAFTNPTTNSYKRLVPGFEAPVMLAYSARNRSASIRIPVVASTKARRIEVRFPDPAANPYLAFAAQLMAGLDGIINKIHPGDAMDKNLYDLPPEEAKEIPTVAGSLEEALAELDKNREFLTRGGVFTDDAIDGYIELLRSDIQRVRMTPHPLEFEMYYSV

SEQ ID NO: 2 - protein, 373 aa, Cricetulus griseus
  REGION 1..373: note = Wild type Cricetulus griseus glutamine synthetase
MATSASSHLNKNIKQMYLCLPQGEKVQAMYIWVDGTGEGLRCKTRTLDCEPKCVEELPEWNFDGSSTFQSEGSNSDMYLSPVAMFRDPFRRDPNKLVFCEVFKYNRKPAETNLRHSCKRIMDMVSNQHPWFGMEQEYTLMGTDGHPFGWPSNGFPGPQGPYYCGVGADKAYGRDIVEAHYRACLYAGVKITGTNAEVMPAQWEFQIGPCEGIRMGDHLWVARFILHRVCEDFGVIATFDPKPIPGNWNGAGCHTNFSTKAMREENGLKHIEEAIEKLSKRHRYHIRAYDPKGGLDNARRLTGFHETSNINDFSAGVANRSASIRIPRTVGQEKKGYFEDRRPSANCDPFAVTEAIVRTCLLNETGDEPFQYKN

SEQ ID NO: 3 - protein, 469 aa, synthetic construct
  VAR_SEQ 130: note = X is any amino acid except for the original amino acid; Providencia vermicola glutamine synthetase E130X
MSAEHILSLIEEHNVRFIDLRFTDTKGKEQHITIPAHQVDEDFFEEGQMFDGSSFGGWKGINESDMVLMPDPSTAMIDPFFADITLIIRCDILEPGTMQGYGRDPRSISKRAEDFLRSSGIADVVLFGPXPEFFVFDDIRFGNSMHSSYYHIDDIEGAWNTGTKYEGGNKGHRPSVKGGYFPVPPVDSSQDLRSAMCTTMEDLGLVVEAHHHEVATAGQNEIATRFNTMTKKADETQVYKYVVHNVAHAFGKTATFMPKPLVGDNGSGMHCHMSLSKDGVNLFAGDKYGGLSEMALYYIGGIIKHARALNAFTNPTTNSYKRLVPGFEAPVMLAYSARNRSASIRIPVVASTKARRIEVRFPDPAANPYLAFAAQLMAGLDGIINKIHPGDAMDKNLYDLPPEEAKEIPTVAGSLEEALAELDKNREFLTRGGVFTDDAIDGYIELLRSDIQRVRMTPHPLEFEMYYSV

SEQ ID NO: 4 - protein, 469 aa, synthetic construct
  REGION 1..469: note = Providencia vermicola glutamine synthetase E130F
MSAEHILSLIEEHNVRFIDLRFTDTKGKEQHITIPAHQVDEDFFEEGQMFDGSSFGGWKGINESDMVLMPDPSTAMIDPFFADITLIIRCDILEPGTMQGYGRDPRSISKRAEDFLRSSGIADVVLFGPFPEFFVFDDIRFGNSMHSSYYHIDDIEGAWNTGTKYEGGNKGHRPSVKGGYFPVPPVDSSQDLRSAMCTTMEDLGLVVEAHHHEVATAGQNEIATRFNTMTKKADETQVYKYVVHNVAHAFGKTATFMPKPLVGDNGSGMHCHMSLSKDGVNLFAGDKYGGLSEMALYYIGGIIKHARALNAFTNPTTNSYKRLVPGFEAPVMLAYSARNRSASIRIPVVASTKARRIEVRFPDPAANPYLAFAAQLMAGLDGIINKIHPGDAMDKNLYDLPPEEAKEIPTVAGSLEEALAELDKNREFLTRGGVFTDDAIDGYIELLRSDIQRVRMTPHPLEFEMYYSV

SEQ ID NO: 5 - protein, 469 aa, synthetic construct
  REGION 1..469: note = Providencia vermicola glutamine synthetase E130G
MSAEHILSLIEEHNVRFIDLRFTDTKGKEQHITIPAHQVDEDFFEEGQMFDGSSFGGWKGINESDMVLMPDPSTAMIDPFFADITLIIRCDILEPGTMQGYGRDPRSISKRAEDFLRSSGIADVVLFGPGPEFFVFDDIRFGNSMHSSYYHIDDIEGAWNTGTKYEGGNKGHRPSVKGGYFPVPPVDSSQDLRSAMCTTMEDLGLVVEAHHHEVATAGQNEIATRFNTMTKKADETQVYKYVVHNVAHAFGKTATFMPKPLVGDNGSGMHCHMSLSKDGVNLFAGDKYGGLSEMALYYIGGIIKHARALNAFTNPTTNSYKRLVPGFEAPVMLAYSARNRSASIRIPVVASTKARRIEVRFPDPAANPYLAFAAQLMAGLDGIINKIHPGDAMDKNLYDLPPEEAKEIPTVAGSLEEALAELDKNREFLTRGGVFTDDAIDGYIELLRSDIQRVRMTPHPLEFEMYYSV

SEQ ID NO: 6 - protein, 469 aa, synthetic construct
  REGION 1..469: note = Providencia vermicola glutamine synthetase F226Y
MSAEHILSLIEEHNVRFIDLRFTDTKGKEQHITIPAHQVDEDFFEEGQMFDGSSFGGWKGINESDMVLMPDPSTAMIDPFFADITLIIRCDILEPGTMQGYGRDPRSISKRAEDFLRSSGIADVVLFGPEPEFFVFDDIRFGNSMHSSYYHIDDIEGAWNTGTKYEGGNKGHRPSVKGGYFPVPPVDSSQDLRSAMCTTMEDLGLVVEAHHHEVATAGQNEIATRYNTMTKKADETQVYKYVVHNVAHAFGKTATFMPKPLVGDNGSGMHCHMSLSKDGVNLFAGDKYGGLSEMALYYIGGIIKHARALNAFTNPTTNSYKRLVPGFEAPVMLAYSARNRSASIRIPVVASTKARRIEVRFPDPAANPYLAFAAQLMAGLDGIINKIHPGDAMDKNLYDLPPEEAKEIPTVAGSLEEALAELDKNREFLTRGGVFTDDAIDGYIELLRSDIQRVRMTPHPLEFEMYYSV

SEQ ID NO: 7 - protein, 469 aa, synthetic construct
  VAR_SEQ 130: note = X is any amino acid except for the original amino acid; Providencia vermicola glutamine synthetase E130X and F226Y
MSAEHILSLIEEHNVRFIDLRFTDTKGKEQHITIPAHQVDEDFFEEGQMFDGSSFGGWKGINESDMVLMPDPSTAMIDPFFADITLIIRCDILEPGTMQGYGRDPRSISKRAEDFLRSSGIADVVLFGPXPEFFVFDDIRFGNSMHSSYYHIDDIEGAWNTGTKYEGGNKGHRPSVKGGYFPVPPVDSSQDLRSAMCTTMEDLGLVVEAHHHEVATAGQNEIATRYNTMTKKADETQVYKYVVHNVAHAFGKTATFMPKPLVGDNGSGMHCHMSLSKDGVNLFAGDKYGGLSEMALYYIGGIIKHARALNAFTNPTTNSYKRLVPGFEAPVMLAYSARNRSASIRIPVVASTKARRIEVRFPDPAANPYLAFAAQLMAGLDGIINKIHPGDAMDKNLYDLPPEEAKEIPTVAGSLEEALAELDKNREFLTRGGVFTDDAIDGYIELLRSDIQRVRMTPHPLEFEMYYSV

SEQ ID NO: 8 - protein, 469 aa, synthetic construct
  REGION 1..469: note = Providencia vermicola glutamine synthetase E130F and F226Y
MSAEHILSLIEEHNVRFIDLRFTDTKGKEQHITIPAHQVDEDFFEEGQMFDGSSFGGWKGINESDMVLMPDPSTAMIDPFFADITLIIRCDILEPGTMQGYGRDPRSISKRAEDFLRSSGIADVVLFGPFPEFFVFDDIRFGNSMHSSYYHIDDIEGAWNTGTKYEGGNKGHRPSVKGGYFPVPPVDSSQDLRSAMCTTMEDLGLVVEAHHHEVATAGQNEIATRYNTMTKKADETQVYKYVVHNVAHAFGKTATFMPKPLVGDNGSGMHCHMSLSKDGVNLFAGDKYGGLSEMALYYIGGIIKHARALNAFTNPTTNSYKRLVPGFEAPVMLAYSARNRSASIRIPVVASTKARRIEVRFPDPAANPYLAFAAQLMAGLDGIINKIHPGDAMDKNLYDLPPEEAKEIPTVAGSLEEALAELDKNREFLTRGGVFTDDAIDGYIELLRSDIQRVRMTPHPLEFEMYYSV

SEQ ID NO: 9 - protein, 469 aa, synthetic construct
  REGION 1..469: note = Providencia vermicola glutamine synthetase E130G and F226Y
MSAEHILSLIEEHNVRFIDLRFTDTKGKEQHITIPAHQVDEDFFEEGQMFDGSSFGGWKGINESDMVLMPDPSTAMIDPFFADITLIIRCDILEPGTMQGYGRDPRSISKRAEDFLRSSGIADVVLFGPGPEFFVFDDIRFGNSMHSSYYHIDDIEGAWNTGTKYEGGNKGHRPSVKGGYFPVPPVDSSQDLRSAMCTTMEDLGLVVEAHHHEVATAGQNEIATRYNTMTKKADETQVYKYVVHNVAHAFGKTATFMPKPLVGDNGSGMHCHMSLSKDGVNLFAGDKYGGLSEMALYYIGGIIKHARALNAFTNPTTNSYKRLVPGFEAPVMLAYSARNRSASIRIPVVASTKARRIEVRFPDPAANPYLAFAAQLMAGLDGIINKIHPGDAMDKNLYDLPPEEAKEIPTVAGSLEEALAELDKNREFLTRGGVFTDDAIDGYIELLRSDIQRVRMTPHPLEFEMYYSV

SEQ ID NO: 10 - protein, 444 aa, Bacillus subtilis
  REGION 1..444: note = Wild type Bacillus Subtilis glutamine synthetase
MAKYTREDIEKLVKEENVKYIRLQFTDILGTIKNVEIPVSQLGKALDNKVMFDGSSIEGFVRIEESDMYLYPDLNTFVIFPWTAEKGKVARFICDIYNPDGTPFEGDPRNNLKRILKEMEDLGFSDFNLGPEPEFFLFKLDEKGEPTLELNDKGGYFDLAPTDLGENCRRDIVLELEEMGFEIEASHHEVAPGQHEIDFKYAGAVRSCDDIQTFKLVVKTIARKHGLHATFMPKPLFGVNGSGMHCNLSLFKNGVNAFFDENADLQLSETAKHFIAGIVKHATSFTAVTNPTVNSYKRLVPGYEAPCYVAWSAQNRSPLIRIPASRGISTRVEVRSVDPAANPYLALSVLLAAGLDGIKNKLEAPAPIDRNIYVMSKEERMENGIVDLPATLAEALEEFKSNEVMVKALGEHLFEHFIEAKEIEWDMFRTQVHPWEREQYMSQY

SEQ ID NO: 11 - protein, 454 aa, Methanocaldococcus jannaschii
  REGION 1..454: note = Wild type Methanocaldococcus jannaschii glutamine synthetase
MHWGMEMNVEQAIEYVKKNNVKFIRFQFVDILGFPKNVAYPVKAGEKGIEELREIFENGVWFDGSSITGFVGIEESDMLLKPDLSTLSVLPWRPEEKSVARVICDVYKDEKTPFEGDPRSRLKAILEELKKEMNGEYFVGPEPEFFLLKRDPHNPHRWVPADDGGYFDVEPLDDAPDIRRDIVLALENLGFHVEASHHEVAPGQHEVDFKFDNALKTADSVITFKMTIKNIAKKHGLKATFMPKPFFGMNGNGMHCHQSVWFNGEPSFYDPEGPYNGLSETCLSYIAGILSHAKALVAITNPTVNSYKRLVPGYEAPVNIAWANKNRSAIIRVPAARGKATRIEFRAPDPTCNPYLAFACMLAAGLDGIKKKMTAPEPVERNIFKMSEEEKKQLGIESVPANLAAALDELECDEVLQKALGKHIYENYMEIKRAEWDDFRTAVTDWETGKYLIY

SEQ ID NO: 12 - protein, 477 aa, Corynebacterium glutamicum
  REGION 1..477: note = Wild type Corynebacterium glutamicum glutamine synthetase
MAFETPEEIVKFIKFIKVEFVDVRFTDLPGTEQHFSIPAASFDADTIEEGLAFDGSSIRGFTRIDESDMNLLPDLGTATLDPFRKAKTLNVKFFFHDPFTREAFSRDPRNVARKAEQYLASTGIADTCNFGAEAEFYLFDSVRYSTEMNSGFYEVDTEEGWWNRGKETNLDGTPNLGAKNRVKGGYFPVAPYDQTVDVRDDMVRNLAASGFALERFHHEVGGGQQEINYRFNTMLHAADDIQTFKYIIKNTARLHGKAATFMPKPLAGDNGSGMHAHQSLWKDGKPLFHDESGYAGLSDIARYYIGGILHHAGAVLAFTNATLNSYHRLVPGFEAPINLVYSQRNRSAAVRIPITGSNPKAKRIEFRAPDPSGNPYLGFAAMMMAGLDGIKNRIEPHAPVDKDLYELPPEEAASIPQAPTSLEASLKALQEDTDYPHRSDVFTEDLIEAYIQYKYDNEISPVRLRPTPQEFELYFDC

SEQ ID NO: 13 - protein, 474 aa, Cryobacterium soli
  REGION 1..474: note = Wild type Cryobacterium soli glutamine synthetase
MFRDSSEVLKFIKDTDVKFLDIRFTDLPGVQQHFNIPASTVDEEFFTVGQMFDGSSIRGFASIHESDMQLIPDVTTAYIDAFRTERTLIMLFDIYNPRNGEIYSKDPRQVAKKAEKYLASTGIADTAFFAPEAEFYIFDDVRYEVNQHSSFYSVDSSEGAWNSGKEEVGGNLANKTPFKGGYFPVSPVDQHADLRDDICLKLIDAGLILERSHHEVGTGGQGEINYRFDTMVHAADDLLKFKYIVKNTSAEWGKTATFMPKPLFGDNGSGMHTHQSLWADGKPLFYDEAGYGGLSDVARWYIGGLLKHAAAVLAFTNPSVNSYHRLVPGFEAPVNLVYSAGNRSASIRIPITGTNPKAKRIEFRAPDASGNPYLAFAAQLMAGLDGIKNRIEPHEPVDKDLYELPPEEAKNIPQVPASLGEALTALENDHDFLLAGNVFTPELIETWIDYKREMEIKPLAQRPHPFEFELYYGV

SEQ ID NO: 14 - protein, 469 aa, Photorhabdus luminescens
  REGION 1..469: note = Wild type Photorhabdus luminescens glutamine synthetase
MSVEHVLTMIEEHQVKFIDLRFTDTKGKEQHMTIPAHQVNADFFEDGKMFDGSSISGWKGINESDMVLMPDTSTAMLDPFFDDATLILRCDILEPGTMQGYDRDPRSISKRAENFLRSSGIADTVLCGPEPEFFLFDDVRFSSAMSHSYYHIDDIEAAWNSGTQYEGGNKGHRPAVKGGYAPLPPVDSSQDLRSAMCLTMEEMGLVVEAHHHEVGTAGQNEIATRFNTMTKKADETQIYKYVVHNVAHNFGKTATFMPKPLVGDNGSGMHCHMSLSKNGTNLFAGDKYGGLSELALFYIGGIIKHARALNAFTNPTTNSYKRLVPGYEAPVMLAYSARNRSASIRIPVVASTKARRIEIRFPDPAANPYLAFAAQLMAGLDGIINKIHPGDAMDKNLYDLPPEEAKEIPTVASSLEDALAALDADREFLTRGGVFTDDAIDAYIDLKNSEIERVRLTPHPLEFELYYSV

SEQ ID NO: 15 - protein, 469 aa, Moellerella wisconsensis
  REGION 1..469: note = Wild type Moellerella wisconsensis glutamine synthetase
MSAEHVLSLIEEHKIRFIDMRFTDTKGKEQHITIPAHQVNEDFFEEGKMFDGSSIGGWKGINESDMVLMPDPSTAMLDPFFVDPTLIIRCDILEPGTMQGYDRDPRSISKRAEDYLRSSGIGDTVLVGPEPEFFVFDDIRFGSTMSGSYYHIDDIEAAWNSGTKYEGGNKGHRPGVKGGYFPVPPVDSSQDMRSAMCTTMEEMGLVVEAHHHEVATAGQNEIATRFNTMTKKADETQIYKYVVHNVAHAYGKTATFMPKPLVGDNGSGMHCHMSIAKDGVNLFAGDKYGGLSEMALYYIGGIIKHARALNAFTNPTTNSYKRLVPGYEAPVMLAYSARNRSASIRIPVVASTKARRIEIRFPDPAANPYLAFAAQLMAGLDGIINKIHPGDAMDKNLYDLPPEEAKEIPTVANSLEDALAELDNDREFLTRGGVFTDDSIDAYIELLRSDIQRVRMTPHPLEFEMYYSV

SEQ ID NO: 16 - protein, 469 aa, Edwardsiella sp.
  REGION 1..469: note = Wild type Edwardsiella glutamine synthetase
MSAEHILAMLNEHEVKFVDLRFTDTKGKEQHVTIPAHQVDADFFEEGKMFDGSSIGGWKGINESDMVLMPDPTTAVLDPFYEEPTLIIRCDILEPGTLQGYDRDPRSISKRAEDFLRASGIADTVLFGPEPEFFLFDDIRFGSSINGSHVAIDDIEGAWNSSTKYEGGNKGHRPAVKGGYFPVPPVDSAQDIRSTMCLTMEEMGLVVEAHHHEVATAGQNEVATRFNTMTKKADEIQIYKYVVHNVAHAFGKTATFMPKPMFGDNGSGMHCHMSLSKNGSNLFAGDKYGGLSETALYYIGGVIKHAKAINALANPTTNSYKRLVPGYEAPVMLAYSARNRSASIRIPVVASPKARRIEVRFPDPAANPYLCFAALLMAGLDGVINKIHPGDAMDKNLYDLPPEEAKEIPTVAGSLDEALAALDSDREFLTRGGVFTDDAIDAYIELRQSEMERVRMTPHPVEFELYYSV

SEQ ID NO: 17 - protein, 469 aa, Budvicia aquatica
  REGION 1..469: note = Wild type Budvicia aquatica glutamine synthetase
MSTEHVLTMLNEHEVKFVDLRFTDTKGKEQHITIPAHQVDADFFEDGKMFDGSSIGGWKGINESDMVLMPDPTTALLDPFFDDTTLIIRCDVLEPGTMQGYERDPRSISKRAEDFLRSSGIADTVLFGPEPEFFLFDDIRFGNSIAGAYYHIDDIEAAWNTGTKYEGGNKGHRPMVKGGYFPVPPVDSSQDIRSAMCLTMEEMGLVVEAHHHEVATAGQNEVATRFNTMTKKADETQIYKYVVHNVAHAFGKTATFMPKPLVGDNGSGMHCHMSLSKNGTNLFAGDKYGGLSEMALYYIGGIIKHAKAINAFANPTTNSYKRLVPGYEAPVMLAYSARNRSASIRIPVVASPKARRIEVRFPDPAANPYLAFAAQLMAGLDGIINKIHPGEAMDKNLYDLPAEEAKNIPTVAGSLEEALASLDADREFLTRGGVFTDDSIDAYIELKQDEMNRVRMAPHPLEFELYYSV

SEQ ID NO: 18 - protein, 469 aa, Brenneria roseae
  REGION 1..469: note = Wild type Brenneria roseae glutamine synthetase
MSAEHVLTMLNEHEVKFVDLRFTDTKGKEQHVTIPAHQVNADFFEEGKMFDGSSIGGWKGINESDMVLMPDASTAVIDPFFEESTLIIRCDILEPGTMQGYDRDPRSISKRAEDFLRSSGIADTVLFGPEPEFFLFDDVRFGSNISGSHVAIDDIEGAWNSGTKYEGGNKGHRPSVKGGYFPVPPVDSSQDLRSTMCLTMEEMGLVVEAHHHEVATAGQNEVATRFNTMTKKADEIQIYKYVVHNVAHAFGKTATFMPKPIFGDNGSGMHCHMSLAKNGTNLFAGDKYGGLSETALFYIGGIIKHAKAINALSNPTTNSYKRLVPGYEAPVMLAYSARNRSASIRIPVVASPKARRIEVRFPDPAANPYLCFSALLMAGLDGVINKIHPGDAMDKNLYDLPPEEASEIPTVAGSLEEALNALDADREFLTRGGVFTDDAINAYIELRKAENDRIRMTPHPVEFELYYSV

SEQ ID NO: 19 - protein, 469 aa, Arsenophonus sp.
  REGION 1..469: note = Wild type Arsenophonus sp. glutamine synthetase
MSVEHVLSMIKEHHVKYIDLRFTDTRGKEQHITIPAHQINQDFFEEGKMFDGSSIAGWKGINESDMMLMPDPETAVLDPFFSDSTLIIRCDILEPDTHKGYERDPRSISKRAENFLRSSGIADTILFGPEPEFFVFDDIRFGNNISDAYYHIDDVEAAWNSGAHYEDGNKGHRPSIKGGYFPVPPVDSSQNMRSAMCSTMEKMGLIVEAHHHEVATAGQNEVATRFNTMTKKADETQIYKYVVHNIAHSFGKTATFMPKPLVGDNGSGMHCHMSLEKNGSNLFAGDKYGGLSEMALYYIGGIIKHARALNAFTNPTTNSYKRLVPGFEAPVMLAYSARNRSASIRIPVVANLRASRIEVRFPDPAANPYLAFAAQLMAGLDGIINKIHPGEAMDKNLYDLPAEEAQEIPTVANSLEEALQALDADREFLKRGEVFTDETIDAYINLLKDDVQRIRMAPHPLEFEIYYSV

SEQ ID NO: 20 - protein, 469 aa, synthetic construct
  REGION 1..469: note = Providencia verimicola glutamine synthetase R345R
MSAEHILSLIEEHNVRFIDLRFTDTKGKEQHITIPAHQVDEDFFEEGQMFDGSSFGGWKGINESDMVLMPDPSTAMIDPFFADITLIIRCDILEPGTMQGYGRDPRSISKRAEDFLRSSGIADVVLFGPEPEFFVFDDIRFGNSMHSSYYHIDDIEGAWNTGTKYEGGNKGHRPSVKGGYFPVPPVDSSQDLRSAMCTTMEDLGLVVEAHHHEVATAGQNEIATRFNTMTKKADETQVYKYVVHNVAHAFGKTATFMPKPLVGDNGSGMHCHMSLSKDGVNLFAGDKYGGLSEMALYYIGGIIKHARALNAFTNPTTNSYKRLVPGFEAPVMLAYSARNRSASIAIPVVASTKARRIEVRFPDPAANPYLAFAAQLMAGLDGIINKIHPGDAMDKNLYDLPPEEAKEIPTVAGSLEEALAELDKNREFLTRGGVFTDDAIDGYIELLRSDIQRVRMTPHPLEFEMYYSV

SEQ ID NO: 21 - protein, 469 aa, synthetic construct
  REGION 1..469: note = Providencia vermicola glutamine synthetase F226Y and R345A
MSAEHILSLIEEHNVRFIDLRFTDTKGKEQHITIPAHQVDEDFFEEGQMFDGSSFGGWKGINESDMVLMPDPSTAMIDPFFADITLIIRCDILEPGTMQGYGRDPRSISKRAEDFLRSSGIADVVLFGPEPEFFVFDDIRFGNSMHSSYYHIDDIEGAWNTGTKYEGGNKGHRPSVKGGYFPVPPVDSSQDLRSAMCTTMEDLGLVVEAHHHEVATAGQNEIATRYNTMTKKADETQVYKYVVHNVAHAFGKTATFMPKPLVGDNGSGMHCHMSLSKDGVNLFAGDKYGGLSEMALYYIGGIIKHARALNAFTNPTTNSYKRLVPGFEAPVMLAYSARNRSASIAIPVVASTKARRIEVRFPDPAANPYLAFAAQLMAGLDGIINKIHPGDAMDKNLYDLPPEEAKEIPTVAGSLEEALAELDKNREFLTRGGVFTDDAIDGYIELLRSDIQRVRMTPHPLEFEMYYSV

SEQ ID NO: 22 - protein, 469 aa, synthetic construct
  VAR_SEQ 130: note = X is any amino acid except for the original amino acid; Providencia vermicola glutamine synthetase E130X and R345A
MSAEHILSLIEEHNVRFIDLRFTDTKGKEQHITIPAHQVDEDFFEEGQMFDGSSFGGWKGINESDMVLMPDPSTAMIDPFFADITLIIRCDILEPGTMQGYGRDPRSISKRAEDFLRSSGIADVVLFGPXPEFFVFDDIRFGNSMHSSYYHIDDIEGAWNTGTKYEGGNKGHRPSVKGGYFPVPPVDSSQDLRSAMCTTMEDLGLVVEAHHHEVATAGQNEIATRFNTMTKKADETQVYKYVVHNVAHAFGKTATFMPKPLVGDNGSGMHCHMSLSKDGVNLFAGDKYGGLSEMALYYIGGIIKHARALNAFTNPTTNSYKRLVPGFEAPVMLAYSARNRSASIAIPVVASTKARRIEVRFPDPAANPYLAFAAQLMAGLDGIINKIHPGDAMDKNLYDLPPEEAKEIPTVAGSLEEALAELDKNREFLTRGGVFTDDAIDGYIELLRSDIQRVRMTPHPLEFEMYYSV

SEQ ID NO: 23 - protein, 469 aa, synthetic construct
  VAR_SEQ 130: note = X is any amino acid except for the original amino acid; Photorhabdus luminescense glutamine synthetase E130X
MSVEHVLTMIEEHQVKFIDLRFTDTKGKEQHMTIPAHQVNADFFEDGKMFDGSSISGWKGINESDMVLMPDTSTAMLDPFFDDATLILRCDILEPGTMQGYDRDPRSISKRAENFLRSSGIADTVLCGPXPEFFLFDDVRFSSAMSHSYYHIDDIEAAWNSGTQYEGGNKGHRPAVKGGYAPLPPVDSSQDLRSAMCLTMEEMGLVVEAHHHEVGTAGQNEIATRFNTMTKKADETQIYKYVVHNVAHNFGKTATFMPKPLVGDNGSGMHCHMSLSKNGTNLFAGDKYGGLSELALFYIGGIIKHARALNAFTNPTTNSYKRLVPGYEAPVMLAYSARNRSASIRIPVVASTKARRIEIRFPDPAANPYLAFAAQLMAGLDGIINKIHPGDAMDKNLYDLPPEEAKEIPTVASSLEDALAALDADREFLTRGGVFTDDAIDAYIDLKNSEIERVRLTPHPLEFELYYSV

SEQ ID NO: 24 - protein, 469 aa, synthetic construct
  REGION 1..469: note = Photorhabdus luminescens glutamine synthetase F226Y
MSVEHVLTMIEEHQVKFIDLRFTDTKGKEQHMTIPAHQVNADFFEDGKMFDGSSISGWKGINESDMVLMPDTSTAMLDPFFDDATLILRCDILEPGTMQGYDRDPRSISKRAENFLRSSGIADTVLCGPEPEFFLFDDVRFSSAMSHSYYHIDDIEAAWNSGTQYEGGNKGHRPAVKGGYAPLPPVDSSQDLRSAMCLTMEEMGLVVEAHHHEVGTAGQNEIATRYNTMTKKADETQIYKYVVHNVAHNFGKTATFMPKPLVGDNGSGMHCHMSLSKNGTNLFAGDKYGGLSELALFYIGGIIKHARALNAFTNPTTNSYKRLVPGYEAPVMLAYSARNRSASIRIPVVASTKARRIEIRFPDPAANPYLAFAAQLMAGLDGIINKIHPGDAMDKNLYDLPPEEAKEIPTVASSLEDALAALDADREFLTRGGVFTDDAIDAYIDLKNSEIERVRLTPHPLEFELYYSV

SEQ ID NO: 25 - protein, 469 aa, synthetic construct
  REGION 1..469: note = Photorhabdus luminescens glutamine synthetase R345A
MSVEHVLTMIEEHQVKFIDLRFTDTKGKEQHMTIPAHQVNADFFEDGKMFDGSSISGWKGINESDMVLMPDTSTAMLDPFFDDATLILRCDILEPGTMQGYDRDPRSISKRAENFLRSSGIADTVLCGPEPEFFLFDDVRFSSAMSHSYYHIDDIEAAWNSGTQYEGGNKGHRPAVKGGYAPLPPVDSSQDLRSAMCLTMEEMGLVVEAHHHEVGTAGQNEIATRFNTMTKKADETQIYKYVVHNVAHNFGKTATFMPKPLVGDNGSGMHCHMSLSKNGTNLFAGDKYGGLSELALFYIGGIIKHARALNAFTNPTTNSYKRLVPGYEAPVMLAYSARNRSASIAIPVVASTKARRIEIRFPDPAANPYLAFAAQLMAGLDGIINKIHPGDAMDKNLYDLPPEEAKEIPTVASSLEDALAALDADREFLTRGGVFTDDAIDAYIDLKNSEIERVRLTPHPLEFELYYSV

SEQ ID NO: 26 - protein, 469 aa, synthetic construct
  VAR_SEQ 130: note = X is any amino acid except for the original amino acid; Photorhabdus luminescens glutamine synthetase E130X and F226Y
MSVEHVLTMIEEHQVKFIDLRFTDTKGKEQHMTIPAHQVNADFFEDGKMFDGSSISGWKGINESDMVLMPDTSTAMLDPFFDDATLILRCDILEPGTMQGYDRDPRSISKRAENFLRSSGIADTVLCGPXPEFFLFDDVRFSSAMSHSYYHIDDIEAAWNSGTQYEGGNKGHRPAVKGGYAPLPPVDSSQDLRSAMCLTMEEMGLVVEAHHHEVGTAGQNEIATRYNTMTKKADETQIYKYVVHNVAHNFGKTATFMPKPLVGDNGSGMHCHMSLSKNGTNLFAGDKYGGLSELALFYIGGIIKHARALNAFTNPTTNSYKRLVPGYEAPVMLAYSARNRSASIRIPVVASTKARRIEIRFPDPAANPYLAFAAQLMAGLDGIINKIHPGDAMDKNLYDLPPEEAKEIPTVASSLEDALAALDADREFLTRGGVFTDDAIDAYIDLKNSEIERVRLTPHPLEFELYYSV

SEQ ID NO: 27 - protein, 469 aa, synthetic construct
  VAR_SEQ 130: note = X is any amino acid except for the original amino acid; Photorhabdus luminescens glutamine synthetase E130X and R345A
MSVEHVLTMIEEHQVKFIDLRFTDTKGKEQHMTIPAHQVNADFFEDGKMFDGSSISGWKGINESDMVLMPDTSTAMLDPFFDDATLILRCDILEPGTMQGYDRDPRSISKRAENFLRSSGIADTVLCGPXPEFFLFDDVRFSSAMSHSYYHIDDIEAAWNSGTQYEGGNKGHRPAVKGGYAPLPPVDSSQDLRSAMCLTMEEMGLVVEAHHHEVGTAGQNEIATRFNTMTKKADETQIYKYVVHNVAHNFGKTATFMPKPLVGDNGSGMHCHMSLSKNGTNLFAGDKYGGLSELALFYIGGIIKHARALNAFTNPTTNSYKRLVPGYEAPVMLAYSARNRSASIAIPVVASTKARRIEIRFPDPAANPYLAFAAQLMAGLDGIINKIHPGDAMDKNLYDLPPEEAKEIPTVASSLEDALAALDADREFLTRGGVFTDDAIDAYIDLKNSEIERVRLTPHPLEFELYYSV

SEQ ID NO: 28 - protein, 469 aa, synthetic construct
  VAR_SEQ 1..469: note = Photorhabdus luminescens glutamine synthetase F226Y and R345A
MSVEHVLTMIEEHQVKFIDLRFTDTKGKEQHMTIPAHQVNADFFEDGKMFDGSSISGWKGINESDMVLMPDTSTAMLDPFFDDATLILRCDILEPGTMQGYDRDPRSISKRAENFLRSSGIADTVLCGPEPEFFLFDDVRFSSAMSHSYYHIDDIEAAWNSGTQYEGGNKGHRPAVKGGYAPLPPVDSSQDLRSAMCLTMEEMGLVVEAHHHEVGTAGQNEIATRYNTMTKKADETQIYKYVVHNVAHNFGKTATFMPKPLVGDNGSGMHCHMSLSKNGTNLFAGDKYGGLSELALFYIGGIIKHARALNAFTNPTTNSYKRLVPGYEAPVMLAYSARNRSASIAIPVVASTKARRIEIRFPDPAANPYLAFAAQLMAGLDGIINKIHPGDAMDKNLYDLPPEEAKEIPTVASSLEDALAALDADREFLTRGGVFTDDAIDAYIDLKNSEIERVRLTPHPLEFELYYSV

SEQ ID NO: 29 - protein, 469 aa, synthetic construct
  VAR_SEQ 130: note = X is any amino acid except for the original amino acid; Providencia vermicola glutamine synthetase E130X and F226Y and R345A
MSAEHILSLIEEHNVRFIDLRFTDTKGKEQHITIPAHQVDEDFFEEGQMFDGSSFGGWKGINESDMVLMPDPSTAMIDPFFADITLIIRCDILEPGTMQGYGRDPRSISKRAEDFLRSSGIADVVLFGPXPEFFVFDDIRFGNSMHSSYYHIDDIEGAWNTGTKYEGGNKGHRPSVKGGYFPVPPVDSSQDLRSAMCTTMEDLGLVVEAHHHEVATAGQNEIATRYNTMTKKADETQVYKYVVHNVAHAFGKTATFMPKPLVGDNGSGMHCHMSLSKDGVNLFAGDKYGGLSEMALYYIGGIIKHARALNAFTNPTTNSYKRLVPGFEAPVMLAYSARNRSASIAIPVVASTKARRIEVRFPDPAANPYLAFAAQLMAGLDGIINKIHPGDAMDKNLYDLPPEEAKEIPTVAGSLEEALAELDKNREFLTRGGVFTDDAIDGYIELLRSDIQRVRMTPHPLEFEMYYSV

SEQ ID NO: 30 - protein, 469 aa, synthetic construct
  VAR_SEQ 130: note = X is any amino acid except for the original amino acid; Photorhabdus luminescens glutamine synthetase E130X and F226Y and R345A
MSVEHVLTMIEEHQVKFIDLRFTDTKGKEQHMTIPAHQVNADFFEDGKMFDGSSISGWKGINESDMVLMPDTSTAMLDPFFDDATLILRCDILEPGTMQGYDRDPRSISKRAENFLRSSGIADTVLCGPXPEFFLFDDVRFSSAMSHSYYHIDDIEAAWNSGTQYEGGNKGHRPAVKGGYAPLPPVDSSQDLRSAMCLTMEEMGLVVEAHHHEVGTAGQNEIATRYNTMTKKADETQIYKYVVHNVAHNFGKTATFMPKPLVGDNGSGMHCHMSLSKNGTNLFAGDKYGGLSELALFYIGGIIKHARALNAFTNPTTNSYKRLVPGYEAPVMLAYSARNRSASIAIPVVASTKARRIEIRFPDPAANPYLAFAAQLMAGLDGIINKIHPGDAMDKNLYDLPPEEAKEIPTVASSLEDALAALDADREFLTRGGVFTDDAIDAYIDLKNSEIERVRLTPHPLEFELYYSV